(19) United States
(12) Patent Application Publication
(10) Pub. No.: US 2006/0229299 A1
(43) Pub. Date: Oct. 12, 2006

(54) 5ht2c Receptor antagonists in the treatment of schizophrenia

(76) Inventor: Anne T. Bruinvels, London (GB)

Correspondence Address: NIXON & VANDERHYE, PC, 901 NORTH GLEBE ROAD, 11TH FLOOR, ARLINGTON, VA, 22203, US

(21) Appl. No.: 10/550,145

(22) PCT Filed: Mar. 19, 2004
(86) PCT No.: PCT/GB04/01225

§ 371 Date: Sep. 21, 2005

(30) Foreign Application Priority Data
Mar. 21, 2003 (GB) 0306604.0

(51) Int. Cl. A61K 31/5513; A61K 31/551; A61K 31/55; A61K 31/5415; A61K 31/405; A61K 31/138

(52) U.S. Cl. 514/220; 514/259.41; 514/419; 514/221; 514/225.8; 514/651

(57) ABSTRACT

The present invention relates to the use of certain 5-HT2C receptor antagonists in the manufacture of medicaments for the treatment of mental disorders, in particular aspects of schizophrenia, cognitive impairment and suicidality, as well as to methods for determining the suitability of compounds for such a use.

The present invention relates to the use of certain compounds for the treatment of mental disorders, in particular schizophrenia, and to methods for determining the suitability of compounds for such a use.

### BACKGROUND TO THE INVENTION

Schizophrenia, a devastating mental disorder, is a chronic disease characterised by severe psychological symptoms such as perception (hallucinations), ideation, reality testing (delusions), thought processes (loose associations), feeling (flatness, inappropriate effect), behaviour (catatonia, disorganisation), attention, concentration, motivation (avolition, impaired intentions and planning) and judgement (see for example Diagnostic and Statistical Manual of Mental Disorders IV, American Psychiatric Association). In general the disease symptoms are divided into positive and negative symptoms with hallucinations and delusions being positive features and features such as flatness, poverty of speech and impaired executive functions representing negative symptoms. Clinical rating scales such as Positive and Negative Syndrome Scale (PANSS, Kay 1991) and Scale for the Assessment of Negative Symptoms (SANS, Andreasen 1982) provide criteria to differentiate between, and rate, positive and negative symptoms. Frequently included in the description of negative symptoms are the cognitive deficits schizophrenic and schizotypical patients suffer from. These include impairment in attention, verbal fluency, executive functions such as planning, working memory and visual and verbal learning and memory. These types of cognitive dysfunction can be measured with a variety of tests, such as Visual Search (Portnoff et al. 1981, Kurachi et al. 1994), Verbal Fluency, Wisconsin Card Sorting, Trail Making—Part B (see Goldberg et al. 1988), Symbol Digit, Hopkins Verbal Learning, Digit Span, Stroop-Color-Word and Attentional Capacity (see Mahurin et al. 1998). Importantly, it has been found that cognitive measures predict work function and overall outcome as assessed by the Global Assessment Scale and Quality of Life Scale (see Meltzer et al. 1996). Several studies have now demonstrated that neuropsychological functions, reflecting several negative and cognitive symptoms of the disease, may be more impaired in male schizophrenic patients when compared to female patients (see Goldstein et al. 1998, 1994, Goldstein and Link, 1988). Further, there are a number of other psychiatric diseases such as schizotypical and schizoaffective disorder, other acute- and chronic psychoses and bipolar disorder which have an overlapping symptomatology with schizophrenia.

Suicide is the major cause of premature death in patients with schizophrenia, with 40% of patients reporting suicidal thoughts, 2 to 40% making unsuccessful suicide attempts and 9% to 13% ending their lives in suicide (Siris, 2001, Meltzer, 1998). Although the pathophysiology of suicide and suicidality remains unclear, the 5-HT2A receptor has been associated with suicide (Du et al, 2001, Pandley et al, 1997). A recent report (Niswender et al, 2001) describes altered levels of an edited from of 5-HT2C receptor messenger RNA in suicide victims.

The aetiology of schizophrenia is still poorly understood and the causes of the disease are thought to be multifactorial. Evidence has been generated to support the fact that schizophrenia is partially a genetic disease (see Baron, 2001, Bassett et al. 2001, Tsuang et al. 2001). The two most reproducible associations with schizophrenia are 1) homozygosity of a dopamine D3 receptor gene variant and 2) a non-functional polymorphism in the serotonin 5-HT2A receptor gene. It is very likely that environmental effects also play a role in the development of the disease (Tsuang et al. 2001). Thus far, no single gene or factor has been found to definitely associate with schizophrenia although many scientific findings point to a hyperactivity in the prefrontal cortex of schizophrenics and a different neuronal packing in that brain structure. Accordingly, a reduction in the levels of pre-synaptic gene transcripts has been found (for review see Lewis and Lieberman, 2000).

Based on the pharmacological actions of antipsychotic drugs as well as pathophysiological data, several hypotheses have been suggested to explain the aetiology of schizophrenia.

Resulting from the mechanism of action of the first generation of anti-psychotic drugs, the dopamine hypothesis of schizophrenia was proposed (suggesting the disease to be caused by hyperactivity of the dopaminergic system, Carlsson 1988, Seeman and Snyder 1975). As this theory could merely explain the positive symptoms of the disease, other hypotheses have been suggested which could explain both the positive and negative symptomatology of schizophrenia. In particular, the glutamate hypothesis, in view of the activity of the street drug and NMDA receptor antagonist phencyclidine (PCP, angel dust), appears to clarify both positive and negative symptoms of the disease (see Olney et al, 1999). Nowadays, the glutamate transmitter system is considered to play a primary role in the development, and possibly treatment, of negative symptoms of schizophrenia (Olney et al. 1999, Bunney et al. 2000). Also the involvement of GABA-ergic, serotonergic and/or cholinergic neurotransmitter systems have been proposed (see Meltzer 2000, Carlsson et al. 2000, Dean 2000, Lewis et al. 1999). Further, a significant body of evidence points to a possible neurodevelopmental cause of the disease (see Weinberger 2000). Altogether, the origin of schizophrenia and several related psychiatric disorders remains unclear and it is likely that the complex aetiology of the disease involves several neurotransmitter systems.

Schizophrenia and related psychotic disorders are currently treated with a variety of antipsychotic drugs. However, the side-effect profile of such drugs can be severe, with side effects including the frequently described extra-pyramidal syndrome (EPS) which is characterised by dyskinesia, akathasia, dystonia and Parkinsonian syndrome often of an irreversible nature (e.g. Casey et al, 1994). According to Deniker and colleagues (Deniker, 1983) antipsychotic activity coincides with EPS and the two could not be dissociated. This concept was generally accepted and was confirmed when a clear correlation between dopamine D2 receptor blockade and antipsychotic activity was observed (Seeman and Lee, 1975, Creese et al, 1976) and the degree of occupancy of the same dopamine receptor was shown to correlate with severity of EPS (Farde et al, 1992).

Many neuroleptic drugs significantly reduce the positive symptoms of schizophrenia but do not affect the negative symptoms of the disease. Further, around 25% of all schizophrenic patients are treatment-resistant to typical antipsychotics and approximately 20% of all patients demonstrate a very severe side-effect profile and cannot, therefore, be treated with these drugs.

Atypical antipsychotics, such as clozapine, cause little or no EPS (Matz et al, 1974, Kane et al, 1993). It has also been found that clozapine's occupancy of the D2 receptor was lower than that of typical D2 receptor blocking antipsychotics (Farde et al. 1989, Pilowski et al. 1992). Clozapine was the first true atypical antipsychotic which significantly improved negative and cognitive deficits in schizophrenia. Clinical studies have found clozapine to be superior to typical as well as other so-called “atypical” antipsychotic drugs—not only does it not produce EPS but also it does not induce increases in serum prolactin levels and appears to ameliorate negative symptoms of the disease.

Although nowadays many novel antipsychotics are termed “atypical”, the criteria for this definition are not clear cut (see Meltzer et al. 1989 for original criteria). Based on the activity profile of clozapine, clinical criteria for novel atypical antipsychotic drugs should include: 1) superior efficacy not only against positive but also against negative (including cognitive) symptoms; 2) efficacy to treat patients refractory to conventional antipsychotic drugs and 3) limited adverse effects profile including no (or minimal) causation of EPS or tardive dyskinesia and a minimal effect on serum prolactin levels (see Waddington and Quinn, 2000).

Generally, typical neuroleptic drugs are very potent dopamine D2 receptor blockers. Clozapine, typifying “atypical” drugs, interacts with a large number of neurotransmitter receptors and it is thought that interaction with one or a few receptors may be key to its atypical activity. It has much lower affinity for dopamine D2 receptor and interacts with α-adrenoceptors, histamine, serotonin (5-HT), muscarinic acetylcholine receptors in addition to certain dopamine receptors (Table 1). (As the 5-HT receptor nomenclature has changed over the past decades, it is recommended to refer to Hoyer et al. (1994) for a recent review.)

Table 1 represents the pharmacological characteristics (expressed as affinity constants) of a range of typical and “atypical” antipsychotic drugs at several human recombinant neurotransmitter receptors.

To improve the understanding of schizophrenia, genes involved in the mediation of action of effective drug treatments have been studied (pharmacogenomics). For psychiatric diseases, and again specifically for schizophrenia, several receptor and “drug target” genes have been investigated with respect to drug treatment response. Pickar and Rubinow (2001) review the latest data from recent pharmacogenomic studies on clozapine response in schizophrenic patients. Many of the genes encoding the receptors with which clozapine interacts (see Table 1) have been investigated for their role in clozapine treatment response. Particularly dopamine and serotonin (5-HT) receptor genes have been studied. Thus far no consistent associations have been found. Two studies have reported associations between different alleles of the dopamine D3 receptor, but the results of both studies are opposing. Interestingly, several studies (six out of ten) have demonstrated positive associations between allelic variants of the 5-HT2A receptor gene and clozapine response, although the effect is small.

TABLE 1
Pharmacological profiles of typical and “atypical”
antipsychotic drugs*

| | 5-HT_{1A} | 5-HT_{2A} | 5-HT_{2C} | 5-HT₃ | 5-HT₆ | 5-HT₇ |
|---|---|---|---|---|---|---|
| Typical Antipsychotic | | | | | | |
| Chlorpromazine | 5.5⁵ | 8.7⁸ | 7.6¹ | | 8.4² | 7.6² |
| Fluphenazine | <5⁵ | 8.6⁸ | 6.2¹ | | 7.8² | 8.1² |
| Fluspirilene | 7.3⁶ | 8.0⁶ | 5.7⁶ | <5.3⁶ | | 7.5⁶ |
| Haloperidol | 5.6⁵ | 6.7^{6,10} | 5.4¹⁰ | <5.3⁶ | <5.3² | 6.6² |
| | 5.8⁶ | 7.7⁸ | 5.6¹ | | | 6.4⁶ |
| Loxapine | 5.5⁵ | 8.7⁸ | 8.0¹ | | 7.8² | 7.4² |
| Pimozide | | 8.1⁸ | <5¹ | | 7.2² | 9.3² |
| Sulpiride | | <5⁸ | <5¹ | | <5.3² | <5.3² |
| Thioridazine | 6.5⁵ | 8.2⁸ | 7.1¹ | | 7.5⁷ | 7.2² |
| | | | | | 8.2² | |
| Thiothixene | 5.9⁵ | 7.3⁸ | 5.8¹ | | 7.4² | 7.9² |
| “Atypical” Antipsychotic | | | | | | |
| Amperozide | 5.9¹⁹ | 7.9⁸ | 5.9¹ | <5¹⁹ | 7.2² | 6.3² |
| Clozapine | 5.7⁵ | 8.3⁸ | 8.1¹ | 7.2¹³ | 8.0⁷ | 8.2² |
| | 6.9⁶ | 8.0⁶ | 7.9⁶ | 7.0⁶ | 8.4² | 7.7⁶ |
| | | | 8.0¹⁰ | | | 7.7¹⁰ |
| Fluperlapine | | 8.1⁸ | 7.7¹ | | 7.8² | 8.3² |
| Iloperidone | 6.8¹⁸ | 8.3¹⁰ | 7.4¹⁰ | | 7.4¹⁰ | 7.7¹⁰ |
| Olanzapine | 5.6⁶ | 8.8¹³ | 8.2¹⁰ | 7.2¹³ | 8.6² | 7.0² |
| | | | 7.3¹³ | 7.1⁶ | | 6.9⁶ |
| Pipamperone | 5.6⁶ | 8.3⁶ | 6.9⁶ | <5.3⁶ | | 6.8⁶ |
| Risperidone | 6.4⁶ | 9.7⁸ | 7.9¹⁰ | <5.3⁶ | <6⁷ | 8.9² |
| | | 9.3⁶ | 7.2⁶ | | 6.4² | 8.8⁶ |
| Seroquel | 6.5⁶ | 7.1⁶ | 5.9¹⁰ | 6.8⁶ | | 6.5⁶ |
| | | 6.8⁴ | 5.4⁶ | 5.4⁶ | | |
| Sertindole | 6.6⁶ | 10.0¹⁹ | 8.8¹⁹ | 5.5⁶ | 9.13¹⁹ | 8.0¹⁹ |
| Ziprasidone | 7.9⁶ | 8.9⁶ | 7.9⁶ | 5.5⁶ | 6.9¹⁴ | 7.6¹⁴ |
| | | | | | | 8.3⁶ |
| Zotepine | 6.5⁶ | 9.2⁸ | 8.5⁶ | 6.6⁶ | 9.0² | 8.8² |
| | | 8.6⁶ | | | | 8.0⁶ |
| Other Drugs | | | | | | |
| Ritanserin | 6.3¹¹ | 9.6¹¹ | 9.0¹¹ | <5¹¹ | | 7.8¹¹ |

| | D₁ | D₂-S | D₂-L | D₃ | D₄ | D₅ |
|---|---|---|---|---|---|---|
| Typical Antipsychotic | | | | | | |
| Chlorpromazine | 7.3⁹ | 8.5¹¹ | 8.3¹¹ | 8.9³ | 7.4¹² | |
| | | 9.3³ | 8.9³ | | 7.9⁴ | |
| Fluphenazine | 7.8⁹ | | 9.2¹⁸ | | 7.3¹² | |
| | | | | | 8.0⁴ | |
| Fluspirilene | | 8.9⁶ | 9.2⁶ | 8.4⁶ | 8.4⁶ | |
| Haloperidol | 7.3⁹ | 8.7⁶ | 8.7⁶ | 8.1⁶ | 8.3¹² | |
| | 7.1¹⁰ | 9.3³ | 9.2³ | 8.6³ | 8.0^{4,6} | |
| Loxapine | 7.5¹⁸ | | 8.1¹⁸ | | 7.9⁴ | |
| Pimozide | 6.0⁹ | 9.7¹¹ | 9.5¹¹ | 9.3³ | 7.4¹² | |
| | | 9.5³ | 9.1³ | | 7.5⁴ | |
| Sulpiride | <5⁹ | 7.0¹¹ | 7.2¹¹ | 8.1³ | 7.3¹² | |
| | | 8.6³ | 8.1³ | | | |
| Thioridazine | 7.9⁹ | 8.4¹¹ | 8.1¹¹ | 8.6³ | 7.9¹² | |
| | | 8.9³ | 8.6³ | | 8.4⁴ | |
| Thiothixene | 6.5⁹ | | 9.2⁸ | | 7.1⁴ | |
| “Atypical” Antipsychotic | | | | | | |
| Amperozide | 5.1¹⁹ | 6.5³ | 6.4³ | 6.6³ | | |
| Clozapine | 7.4⁹ | 6.8⁶ | 6.7⁶ | 6.6⁶ | 7.4⁶ | 6.6¹⁰ |
| | 6.7¹⁰ | 7.5³ | 7.2³ | 7.1³ | 8.0^{4,12} | |
| | | | | | 7.3¹⁰ | |
| Fluperlapine | 6.8⁸ | | 6.5⁸ | | 6.9⁴ | |
| Iloperidone | 6.7¹⁰ | 7.9¹⁰ | 8.2¹⁰ | 8.1¹⁰ | 7.6¹⁰ | 6.5¹⁰ |
| Olanzapine | 8.1¹³ | 7.7⁶ | 7.5⁶ | 7.3⁶ | 7.6^{6,13} | |
| | 7.5¹⁰ | | | | 8.0⁴ | |
| Pipamperone | 5.6⁶ | 7.0⁶ | 6.9⁶ | 6.6⁶ | 8.3⁶ | |
| Risperidone | 6.3¹⁰ | 8.2⁶ | 8.2⁶ | 7.9⁶ | 7.8⁴ | |
| | | 8.9³ | 8.8³ | 8.2³ | 7.8⁶ | |
| Seroquel | 5.9¹⁰ | 6.4⁶ | 6.2⁶ | 6.5⁶ | 5.8⁶ | |
| | | | | | 5.9⁴ | |
| Sertindole | 6.89¹⁹ | 8.2⁶ | 7.9¹⁹ | 8.0⁶ | 7.6¹⁹ | |
| Ziprasidone | 6.3¹⁴ | 8.4⁶ | 8.4¹⁴ | 8.1¹⁴ | 7.5¹⁴ | |
| | | | 8.3⁶ | 8.0⁶ | 7.4⁶ | |
| Zotepine | 7.5⁹ | 8.3⁶ | 8.0⁶ | 8.2⁶ | 7.4⁶ | |
| | | | | | 8.2⁴ | |
| Other Drugs | | | | | | |
| Ritanserin | 5.9¹¹ | | 7.4¹¹ | 7.1¹¹ | | |

| | M₁ | M₂ | M₃ | M₄ | M₅ | mACh |
|---|---|---|---|---|---|---|
| Typical Antipsychotic | | | | | | |
| Chlorpromazine | 7.6¹⁵ | 6.8¹⁵ | 7.2¹⁵ | 7.4¹⁵ | 7.4¹⁵ | 7.2¹⁶ |
| Fluphenazine | | | | | | 5.3¹⁶ |
| Fluspirilene | | | | | | <5.3⁶ |
| Haloperidol | | | | | | <5¹⁶ |
| Loxapine | 7.2¹⁵ | 6.5¹⁵ | 6.4¹⁵ | 6.5¹⁵ | 6.6¹⁵ | 6.3¹⁶ |
| Pimozide | | | | | | |
| Sulpiride | | | | | | |
| Thioridazine | 8.6¹⁵ | 7.9¹⁵ | 7.8¹⁵ | 8.0¹⁵ | 7.9¹⁵ | 7.7¹⁶ |
| Thiothixene | 5.6¹⁵ | 5.7¹⁵ | 5.8¹⁵ | 5.8¹⁵ | 5.4¹⁵ | 5.5¹⁶ |
| “Atypical” Antipsychotic | | | | | | |
| Amperozide | | | | | | 6.3¹⁹ |
| Clozapine | 8.5¹⁵ | 7.3¹⁵ | 7.7¹⁵ | 8.0¹⁵ | 8.0¹⁵ | 7.9¹⁶ |
| | 8.9¹³ | | 8.2¹³ | 8.2¹³ | 8.3¹³ | |
| Fluperlapine | 8.1¹⁵ | 7.1¹⁵ | 7.4¹⁵ | 7.9¹⁵ | 7.7¹⁵ | |
| Iloperidone | | | | | | <5¹⁷ |
| Olanzapine | 8.6¹³ | | 7.9¹³ | 8.0¹³ | 8.2¹³ | 8.7¹³ |
| | | | | | | 7.6⁶ |
| Pipamperone | | | | | | <5.3⁶ |
| Risperidone | 5.0¹⁵ | 5.4¹⁵ | <5¹⁵ | 5.5¹⁵ | <5¹⁵ | <5.3⁶ |
| Seroquel | 6.9¹³ | | 6.2¹³ | 6.6¹³ | 5.5¹³ | 6.0⁶ |
| Sertindole | | | | | | <5.6¹⁹ |
| Ziprasidone | | | | | | <5.3⁶ |
| | | | | | | <6¹⁴ |
| Zotepine | 7.7¹⁵ | 6.9¹⁵ | 7.1¹⁵ | 7.1¹⁵ | 6.6¹⁵ | 6.3⁶ |
| Other Drugs | | | | | | |
| Ritanserin | | | | | | 5.4¹¹ |

| | α₁ | α₂ | H₁ | sigma |
|---|---|---|---|---|
| Typical Antipsychotic | | | | |
| Chlorpromazine | 8.6¹⁶ | 6.1¹⁶ | 8.0¹⁶ | |
| Fluphenazine | 8.0¹⁶ | 5.8¹⁶ | 7.7¹⁶ | |
| Fluspirilene | 7.0⁶ | <5.3⁶ | 6.3⁶ | 6.8⁶ |
| Haloperidol | 8.2¹⁶ | 5.4¹⁶ | 5.7¹⁶ | 9.0⁶ |
| Loxapine | 7.6¹⁶ | 5.6¹⁶ | 8.3¹⁶ | |
| Pimozide | | | | |
| Sulpiride | | | | |
| Thioridazine | 8.3¹⁶ | 6.1¹⁶ | 7.8¹⁶ | |
| Thiothixene | 8.0¹⁶ | 6.7¹⁶ | 8.2¹⁶ | |
| “Atypical” Antipsychotic | | | | |
| Amperozide | 6.2¹⁹ | 5.5¹⁹ | 6.9¹⁹ | 6.3¹⁹ |
| Clozapine | 8.0¹⁶ | 6.8¹⁶ | 8.6¹⁶ | <5.3⁶ |
| Fluperlapine | | | | |
| Iloperidone | 9.6¹⁷ | 7.5¹⁷ | | 7.5¹⁷ |
| Olanzapine | 7.2¹³ | 6.6¹³ | 8.2¹³ | <5.3⁶ |
| Pipamperone | 7.2⁶ | 6.2⁶ | <5.3⁶ | 7.3⁶ |
| Risperidone | 8.6⁶ | 8.1⁶ | 7.7¹⁴ | 6.0⁶ |
| | | | 8.6⁶ | |
| Seroquel | 7.2⁶ | 6.0⁶ | 6.7⁶ | <5.3⁶ |
| Sertindole | 8.7¹⁹ | 6.5¹⁹ | 6.3¹⁹ | 6.9⁶ |
| Ziprasidone | 8.0¹⁴ | <6 | 7.3¹⁴ | <5.3⁶ |
| Zotepine | 8.5⁶ | 6.0⁶ | 8.5⁶ | 6.3⁶ |
| Other Drugs | | | | |
| Ritanserin | 7.5¹¹ | 7.3¹¹ | 8.2¹¹ | 6.1¹¹ |

*Data are expressed as affinity values (−logKᵢ, or −log K_{D} if indicated) for human receptors, if possible. See note following references for species used.
¹Roth et al, 1992 (rat);
²Roth et al, 1994 (rat);
³Malmberg et al, 1993 (human);
⁴Roth et al, 1995 (rat);
⁵Wander et al, 1987 (human);
⁶Schotte et al, 1996 (5-HT_{1A}-rat, 5-HT_{2A}-human, 5-HT_{2C}-pig, 5-HT₃-mouse, 5-HT₇-mouse, D₁-rat, D_{2/3/4}-human, mACh-rat, H₁-guinea-pig, sigma-guinea-pig);
⁷Glatt et al, 1995 (rat);
⁸Meltzer et al, 1989b (rat);
⁹Kanba et al, 1994 (human);
¹⁰Kongsamut et al, 1996 (all human with exception of rat 5-HT₆ and 5-HT₇);
¹¹Leysen et al. 1993a (human and for ritanserin data also rat);
¹²van Tol et al, 1991 (human);
¹³Bymaster et al, 1996 (human D_{1/2/4}, 5-HT_{2A}, 5-HT_{2C}, m₁-m₅, rat 5-HT₃, α_{1/2}, H₁);
¹⁴Seeger et al, 1995 (all rat with exception of human D_{2/3/4} and H₁);
¹⁵Bolden et al, 1992 (human, expressed as K_{D});
¹⁶Richelson and Nelson, 1984 (human, expressed as K_{D});
¹⁷Szewczak et al, 1995 (rat);
¹⁸Corbett et al, 1993 (rat),
¹⁹Leysen 2000 (human).
Abbreviations used:
5-HT, serotonin receptor;
D, dopamine receptor;
D₂-S/D₂-L, short and long form of D₂ receptor, respectively;
M or mACh, muscarinic acetylcholine receptor;
α, α-adrenoceptor;
H, histamine receptor,
sigma, σ receptor.

There are data to suggest that, of all neurotransmitter receptors targeted by clozapine, the 5-HT2A receptor gene and possibly the D3 receptor gene may play a (limited) role in the susceptibility to the development of schizophrenia and maybe also participate in the response to clozapine treatment.

Other serotonin receptor targets, such as the 5-HT2C receptor (gene), have not been highlighted in these reviews and are believed not to play a significant role in either risk to the disease or response to clozapine treatment (see O'Donovan and Owen, 1999, Baron, 2001, Bassett et al. 2001, Pickar and Rubinow, 2001). Substantial research efforts are currently underway to identify genes or factors which represent a more significant risk factor to schizophrenia and/or related psychiatric disorders.

Although it has been recognised that clozapine and several other effective (atypical) antipsychotics are potent 5-HT2C receptor antagonists, a possible role for 5-HT2C receptor antagonism in antipsychotic medication is generally not favoured. For example, Canton and colleagues (1991) presented data suggesting a correlation between 5-HT2C receptor affinity and efficacy of clozapine (and atypical antipsychotic activity in general). However, Roth and co-workers (1992) presented data demonstrating that high affinity for the 5-HT2C receptor does not correlate with their selection of “atypical” antipsychotics and therefore discarded the hypothesis that 5-HT2C receptor antagonism may be an important component of atypical antipsychotic drugs. Further reviews, which have discussed the putative role of 5-HT2C receptor antagonists in antipsychotic treatments, have primarily concluded that 5-HT2C receptor antagonism 1) does not contribute to antipsychotic activity (Leysen et al. 1993), 2) leads to weight gain (Leysen 2000) and 3) that it functionally opposes 5-HT2A receptor antagonism (Meltzer 1999).

Although several of the new generation of antipsychotic drugs demonstrate limited extra-pyramidal side effects (often only at low doses), not all are devoid of increases of prolactin release (as specified in the original definition). Further, few of the new antipsychotics have been reported to ameliorate negative symptoms or cognitive deficits of the disease.

U.S. Pat. No. 6,335,371 describes a method for inducing cognition enhancement in a mammal by administration of deramciclane and derivatives thereof, these compounds being 5HT2A and/or 5HT2C receptor antagonists.

Several groups including, Altar et al. (1986), Leysen and colleagues (1988, 1993) and Meltzer (1994, 1996, 1999), recognized that many effective antipsychotic drugs have a high 5-HT2A receptor affinity in addition to moderate to high affinity for the D2 receptor. Meltzer et al. reported that specifically “atypical” antipsychotics have a high ratio of 5-HT2A/D2 receptor affinity (Meltzer et al. 1989, reporting rat data). However, the correlation between the 5-HT2A/D2 ratio was not ideal, e.g. loxapine and zotepine were outliers, and it was suggested that it should only be used as a rapid screening tool. Table 2 lists the 5-HT2A/D2 receptor affinity ratios for typical and “atypical” antipsychotics determined mainly at human recombinant receptors and a similar relationship as reported by Meltzer et al. (1989) can be observed.

TABLE 2
Receptor Affinity ratios for several typical and
atypical antipsychotic drugs*

| | | 5-HT_{2C}/ | 5-HT_{2C}/ | | |
|---|---|---|---|---|---|
| | 5- | 5-HT_{2A} | D2 | (2C/2A) + | |
| | HT_{2A}/D₂ | (2C/2A) | (2C/D2) | (2C/D2) | H₁/D₂ |
| Typical | | | | | |
| Antipsychotic | | | | | |
| Chlorpromazine | 1.01 | 0.87 | 0.88 | 1.75 | 0.93 |
| Fluphenazine | 0.93 | 0.72 | 0.67 | 1.39 | 0.84 |
| Fluspirilene | 0.87 | 0.71 | 0.62 | 1.33 | 0.68 |
| Haloperidol | 0.75 | 0.76 | 0.61 | 1.37 | 0.63 |
| Loxapine | 1.07 | 0.92 | 0.99 | 1.91 | 1.02 |
| Pimozide | 0.87 | <0.6 | <0.54 | <1.14 | |
| Sulpiride | <0.65 | <1 | <0.65 | <1.65 | |
| Thioridazine | 0.98 | 0.87 | 0.85 | 1.72 | 0.93 |
| Thiothixene | 0.79 | 0.79 | 0.63 | 1.43 | 0.89 |
| “Atypical” | | | | | |
| Antipsychotic | | | | | |
| Amperozide | 1.23 | 0.76 | 0.92 | 1.67 | 1.08 |
| Clozapine | 1.16 | 0.98 | 1.14 | 2.12 | 1.23 |
| Fluperlapine | 1.25 | 0.95 | 1.18 | 2.13 | |
| Iloperidone | 1.01 | 0.89 | 0.90 | 1.79 | |
| Olanzapine | 1.17 | 0.89 | 1.04 | 1.93 | 1.05 |
| ORG-5222 | 1.10 | 1.02 | 1.13 | 2.15 | 1.02 |
| Pipamperone | 1.20 | 0.83 | 1.0 | 1.83 | <0.77 |
| Risperidone | 1.12 | 0.80 | 0.89 | 1.69 | 0.9 |
| Seroquel | 1.13 | 0.81 | 0.92 | 1.73 | 1.08 |
| Sertindole | 1.27 | 0.89 | 1.11 | 1.99 | 0.79 |
| Ziprasidone | 1.06 | 0.89 | 0.94 | 1.83 | 0.87 |
| Zotepine | 1.08 | 0.96 | 1.06 | 2.02 | 1.06 |
| Other Drugs | | | | | |
| Ritanserin | 1.29 | 0.94 | 1.22 | 2.15 | 1.10 |
| *For the affinity ratios, data from Table 1 have been used During the past decade, the relatively high affinity of atypical antipsychotic drugs for the 5-HT2A receptor combined with lower affinity for D2 receptors has generally been seen as the single most important factor differentiating atypical from typical antipsychotic drugs (Richelson 1996, Leysen et | | | | | |
| # al. 1993, Meltzer 1994, 1996, 1999). Other pharmacological characteristics thought to be of importance in “atypical” antipsychotic activity are dopamine D1 and/or D4 receptor antagonism, possibly serotonin 5-HT1A, 5-HT6 and 5-HT7 receptor antagonism, anticholinergic properties and adrenergic α1-receptor antagonism (Lieberman, 1993, Meltzer, 1994, 1999, Richelson, 1996, Leysen, 2000). | | | | | |

In spite of the extensive studies that have been performed in respect of the treatment of schizophrenia, particularly in the assessment of the relationship between the desirable effects caused by atypical anti-psychotics and receptor antagonism, there remains a need for the identification of candidate drugs which address cognitive deficits, as well as the negative aspects of the condition in particular, but which also address the aspects of the condition which result in suicide.

### SUMMARY OF THE INVENTION

The present invention is based upon the discovery that compounds which antagonise the 5-HT2C receptor are particularly suitable for the treatment of certain groups of schizophrenia sufferers, as well as for the treatment of patients suffering from related disorders. The invention also provides means for determining the suitability of compounds for use in the treatment of schizophrenia and related psychiatric disorders. More particularly, the relative affinity of candidate compounds for the 5-HT2C receptor is assessed and, dependent on that affinity, the suitability of such compounds for the treatment of schizophrenia and related psychiatric disorders is determined.

### DETAILED DESCRIPTION OF THE INVENTION

In its first aspect, the present invention provides the use of a 5-HT2C receptor antagonist in the manufacture of a medicament for the treatment of cognitive dysfunction in and/or negative symptoms of schizophrenia, in the treatment of refractory schizophrenia or in the treatment of suicidality or mild cognitive impairment, with the proviso that:

(a) for the indications cognitive dysfunction, negative symptoms of schizophrenia and refractory schizophrenia, the 5-HT2C receptor antagonist is other than ritanserin, clozapine, fluperlapine, loxapine, ORG-5222, pipamperone, sertindole, olanzapine, zotepine or ziprasidone;

(b) for the indications cognitive dysfunction in schizophrenia or mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and

(c) for the treatment of schizophrenic suicidality, the 5-HT2C receptor antagonist is other than clozapine.

In its second aspect, the present invention provides the use of a compound having a relative 5-HT2C affinity of ≧1.80, wherein the relative 5-HT2C affinity is determined according to formula I:

$$Formula I : \\ \frac{X}{A} + \frac{X}{B}$$

[wherein X is the average affinity of a compound for interaction at the 5-HT2C receptor and A and B are the average affinity values of a compound for interaction at two major sites other than the 5-HT2C receptor] in the preparation of a medicament for the treatment of cognitive dysfunction in and/or negative symptoms of schizophrenia, in the treatment of refractory schizophrenia or in the treatment of suicidality or mild cognitive impairment, with the proviso that:
(a) for the indications cognitive dysfunction, negative symptoms of schizophrenia or refractory schizophrenia, the compound is other than ritanserin, clozapine, fluperlapine, loxapine, ORG-5222, pipamperone, sertindole, olanzapine, zotepine or ziprasidone;
(b) for the indications cognitive dysfunction in schizophrenia or mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and
(c) for the treatment of schizophrenic suicidality, the compound is other than clozapine.

In a third aspect, the present invention provides a method for determining the suitability of a candidate compound for use in the treatment of cognitive dysfunction in and/or negative symptoms of schizophrenia, refractory schizophrenia, suicidality or mild cognitive impairment, which comprises:
a) assessing the affinity of the compound at the 5-HT2C receptor;
b) assessing the affinity of the compound at at least two other major sites of said compound interaction;
c) applying the assessed affinities to the following formula:

$$\frac{X}{A} + \frac{X}{B} = Y$$

[wherein: X is the average affinity of a compound for interaction at the 5-HT2C receptor and A and B are the average affinity values of a compound for interaction at two major sites other than the 5-HT2C receptor]; and selecting compounds in which Y≧1.80 as suitable compounds for the treatment of cognitive dysfunction in and/or negative symptoms of schizophrenia, refractory schizophrenia, suicidality or mild cognitive impairment, provided that:
(a) for the treatment of cognitive dysfunction in or negative symptoms of schizophrenia or refractory schizophrenia, the compound selected is other than ritanserin, clozapine, fluperlapine, loxapine, ORG-5222, pipamperone, sertindole, olanzapine, zotepine or ziprasidone;
(b) for the indications cognitive dysfunction in schizophrenia or mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and
(c) for the treatment of schizophrenic suicidality, the compound selected is other than clozapine.

In a preferred embodiment of this aspect of the invention, there is provided a method for determining the suitability of a candidate compound for use in the treatment of cognitive dysfunction in and/or negative symptoms of schizophrenia or refractory schizophrenia, which comprises:
a) assessing the affinity of the compound at the 5-HT2C receptor;
b) assessing the affinity of the compound at at least two other major sites of said compound interaction;
c) applying the assessed affinities to the following formula:

$$\frac{X}{A} + \frac{X}{B} = Y$$

[wherein: X is the affinity of a compound for interaction at the 5-HT2C receptor and A and B are the average affinity values of a compound for interaction at two major sites other than the 5-HT2C receptor];
(d) and selecting compounds in which Y≧1.80 as suitable compounds for the treatment of cognitive dysfunction in and/or negative symptoms of schizophrenia or refractory schizophrenia, provided (i) that the compound selected is other than ritanserin, clozapine, fluperlapine, loxapine, ORG-5222, pipamperone, sertindole, olanzapine, zotepine or ziprasidone; and (ii) when the compound selected is for use in the treatment of cognitive dysfunction, the compound is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof.

In a further preferred embodiment of this aspect of the invention, there is provided a method for determining the suitability of a candidate compound for use in the treatment of suicidality or mild cognitive impairment, which comprises:
a) assessing the affinity of the compound at the 5-HT2C receptor;
b) assessing the affinity of the compound at at least two other major sites of said compound interaction;
c) applying the assessed affinities to the following formula:

$$\frac{X}{A} + \frac{X}{B} = Y$$

[wherein: X is the affinity of a compound for interaction at the 5-HT2C receptor and A and B are the average affinity values of a compound for interaction at two major sites other than the 5-HT2C receptor];
(e) and selecting compounds in which Y≧1.80 as suitable compounds for the treatment of suicidality or mild cognitive impairment, with the proviso that, (i) for the treatment of schizophrenic suicidality, the compound selected is other than clozapine; and (ii) for the treatment of mild cognitive impairment, the compound is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof.

In another aspect, the present invention provides a method for the treatment of a patient suffering from symptoms associated with a condition selected from the group consisting of negative symptoms of schizophrenia, cognitive dysfunction, refractory schizophrenia, suicidality and mild cognitive impairment with a pharmaceutically effective amount of a 5-HT2C antagonist, with the proviso that:

(a) when the condition is selected from the group consisting of negative symptoms of schizophrenia, cognitive dysfunction and refractory schizophrenia, the 5-HT2C receptor antagonist is other than ritanserin, clozapine, fluperlapine, loxapine, ORG-5222, pipamperone, sertindole, olanzapine, zotepine or ziprasidone;

(b) when the condition is cognitive dysfunction in schizophrenia or is mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and

(c) when the condition is schizophrenic suicidality, the 5-HT2C receptor antagonist is other than clozapine.

In a yet further aspect, the present invention provides a method for the treatment of a patient suffering from symptoms associated with a condition selected from the group consisting of negative symptoms of schizophrenia, cognitive dysfunction, refractory schizophrenia, suicidality and mild cognitive impairment with a pharmaceutically effective amount of a compound having a relative 5-HT2C affinity of ≧1.80, wherein the relative 5-HT2C affinity is determined according to formula I:

$$Formula I : \\ \frac{X}{A} + \frac{X}{B}$$

[wherein X is the average affinity of a compound for interaction at the 5-HT2C receptor and A and B are the average affinity values of a compound for interaction at two major sites other than the 5-HT2C receptor] with the proviso that:
(a) when the condition is selected from the group consisting of cognitive dysfunction, negative symptoms of schizophrenia or refractory schizophrenia, the compound is other than ritanserin, clozapine, fluperlapine, loxapine, ORG-5222, pipamperone, sertindole, olanzapine, zotepine or ziprasidone;
(b) for the indications cognitive dysfunction in schizophrenia and mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and
(c) when the condition is schizophrenic suicidality, the compound is other than clozapine.

In the present invention, A and B are different and can, independently, be any site which binds the compound, including receptors, channels, enzymes or any other protein, such as any of the receptors listed in Table 1.

In a preferred embodiment of these aspects of the invention, A and B are different and are independently selected from the group consisting of the 5-HT1A, 5-HT2A, 5-HT₃, 5-HT₆, 5-HT₇, D₁, D₂-S, D₂-L, D₃, D₄, D₅ M₁, M₂, M₃, M₄, M₅, mACh, α1, α2, H₁ or sigma receptors.

More preferably A and B are different and are independently selected from the group consisting of the 5-HT₃, 5-HT₄, 5-HT₆, 5-HT₇, D₁, D₂, D₃, M₁, M₂, M₃, M₄, M₅, α₁, α₂ or H₁ receptors.

In a most preferred embodiment of these aspects of the invention, A is typically a measurement of the affinity of the compound for the 5-HT2A receptor.

In a further most preferred embodiment of these aspects of the invention, B is typically a measurement of the affinity of the compound for the D2 receptor.

In a more preferred embodiment of these aspects of the invention, A is a measurement of the affinity of the compound for the 5-HT2A receptor and B is a measurement of the affinity of the compound for the D2 receptor.

Affinity of a compound for any of the receptors, channels, enzymes or proteins such as those listed in Table 1 can be measured using techniques common in the art. Typically, affinity is measured as logKi (pKi) or logKd (pKd).

In the present invention, compounds having a value for Y in Formula I greater than or equal to 1.80 are deemed as being particularly suitable for use in the treatment of the specific patient groups of the invention. It is generally the case that the higher the value for Y, the more suitable is the compound for use in the treatment of the specific patient groups detailed herein. In a preferred embodiment of the present invention, Y is equal to or greater than 2.00, although it is most preferred that Y is equal to or greater than 1.90.

5-HT2C receptor antagonists and, in particular such compounds having a relative 5-HT2C affinity ≧1.80 are useful in the treatment of cognitive dysfunction in and/or negative symptoms of schizophrenia, refractory schizophrenia, suicidality or mild cognitive impairment.

Compounds such as ritanserin, clozapine, fluperlapine, loxapine, ORG-5222, pipamperone, sertindole, olanzapine, zotepine or ziprasidone are already known for the treatment of schizophrenia and are hence excluded for the purposes of the present invention for use in the manufacture of a medicament for the treatment of cognitive dysfunction in or negative symptoms of schizophrenia or refractory schizophrenia. Clozapine is also known for the treatment of suicidality in schizophrenic patients and is hence excluded therefrom for the purposes of this invention. As discussed above, U.S. Pat. No. 6,335,371 discloses the use of deramciclane, N-desmethylderamciclane and pharmaceutically acceptable acid addition salts thereof in the treatment of cognitive dysfunction in, i.a., psychiatric disorders and hence the present application excludes this use for these compounds.

Cognitive dysfunction is typified by impairment in attention, verbal fluency, executive functions such as planning, working memory and visual and verbal learning and memory. In some patients with schizophrenia, cognitive functioning declines with impaired attention, abstract thinking and problem solving. Severity of cognitive impairment is a major determinant of overall disability in these patients.

In accordance with the present invention, it has been discovered that 5-HT2C receptor antagonists are useful in the treatment of these symptoms and the present invention therefore provides the use of a 5-HT2C receptor antagonist in the manufacture of a medicament for the treatment of cognitive dysfunction in a schizophrenic patient, with the proviso that the 5-HT2C receptor antagonist is other than ritanserin, clozapine, fluperlapine, loxapine, ORG-5222, pipamperone, sertindole, olanzapine, deramciclane, N-desmethylderamciclane, zotepine or ziprasidone.

Negative (deficit) symptoms of schizophrenia include blunted affect, poverty of speech, anhedonia and asociality. With blunted affect (flattening of emotions), the patient's face may appear immobile, with poor eye contact and lack of expressiveness. Poverty of speech refers to a dimunition of thought reflected in decreased speech and terse replies to questions, creating the impression of inner emptiness. Anhedonia (diminished capacity to experience pleasure) may be reflected by a lack of interest in activities with substantial time being spent in purposeless activity. Asociality refers to a lack of interest in relationships. Negative symptoms are often associated with a general loss of motivation and diminished sense of purpose and goals. Patients with a “deficit subtype” of schizophrenia have prominent negative symptoms unaccounted for by other factors. Such patients are typically more disabled, have a poorer prognosis and are more resistant to treatment than those with a “nondeficit subtype” of schizophrenia.

In accordance with the present invention it has been discovered that 5-HT2C receptor antagonists are useful in the treatment of negative symptoms of schizophrenia, and the present invention therefore provides the use of a 5-HT2C receptor antagonist in the manufacture of a medicament for the treatment of negative symptoms of schizophrenia, with the proviso that the 5-HT2C receptor antagonist is other than ritanserin, clozapine, fluperlapine, loxapine, ORG-5222, pipamperone, sertindole, olanzapine, zotepine or ziprasidone.

Negative symptoms of schizophrenia can be found either together with or in isolation from cognitive dysfunction in schizophrenic patients. Excluded from the present invention is the use of (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof in the manufacture of a medicament for the treatment of cognitive dysfunction in a patient also suffering from negative symptoms of schizophrenia.

Refractory schizophrenia is a term given to embrace those schizophrenic patients who do not respond to conventional antipsychotic drugs. This groups generally makes up approximately 30% of all schizophrenic patients. In accordance with the present invention, it has been discovered that 5-HT2C receptor antagonists are useful in the treatment of refractory schizophrenia and the present invention therefore provides the use of a 5-HT2C receptor antagonist in the manufacture of a medicament for the treatment of refractory schizophrenia, with the proviso that the 5-HT2C receptor antagonist is other than ritanserin, clozapine, fluperlapine, loxapine, ORG-5222, pipamperone, sertindole, olanzapine, zotepine or ziprasidone.

Suiciality may or may not be associated with schizophrenia. Suicidality is often associated with people with personality disorders, particularly emotionally immature people who have a borderline or an antisocial personality disorder, tolerate frustration poorly and react to stress impetuously with violence and aggression.

According to the Merck Manual of Diagnosis and Therapy, 17^{th} Edition (www.merck.com/pubs/mmanual), suicidal behaviour includes suicide gestures, attempted suicide and completed suicide. Suicide plans and actions that appear unlikely to succeed are often termed “suicide gestures”; they are predominantly communicative and are generally pleas for help. It is important to aim treatment at relieving misery and preventing repeated attempts, particularly as 20% of people who attempt suicide try again within 1 year and 10% finally succeed. “Attempted suicide” is a suicidal act that is not fatal, possibly because the self-destructive intention was slight, vague or ambiguous or the action taken had a low lethal potential. Most people who attempt suicide are ambivalent about their wish to die and the attempt may be a plea for help and may fail because of a strong wish to live. “Completed suicide” results in death.

Some patients with schizophrenia attempt suicide and may or may not be successful. In chronic schizophrenia, suicide may result from the episodes of depression to which these patients are prone. The suicide method is usually bizarre and often violent. Attempted suicide is uncommon, although it may be the first gross sign of psychiatric disturbance, occurring early in schizophrenia, possibly when the patient becomes aware of the disorganisation of his thought and volitional processes.

In accordance with the present invention, there is provided the use of a 5-HT2C receptor antagonist in the manufacture of a medicament for the treatment of suicidality, with the proviso that, when the suicidality is in a schizophrenic patient, the 5-HT2C receptor antagonist is other than clozapine. Suicidality may be in a schizophrenic or non-schizophrenic patient and, in a preferred aspect, therefore, the present invention provides the above use of a 5-HT2C receptor antagonist wherein the suicidality is in a schizophrenic patient, with the proviso that the 5-HT2C receptor antagonist is other than clozapine. In an alternatively preferred aspect, the present invention provides the above use of a 5-HT2C receptor antagonist wherein the suicidality is not in a schizophrenic patient.

Mild cognitive impairment is a term given to patients whose clinical state presents as memory impaired, but who are otherwise functioning well and do not meet the clinical criteria for dementia. Mild cognitive impairment represents a transitional state of cognitive impairment between normal aging and early Alzheimer disease. Diagnostic criteria typically include memory complaint (preferably corroborated), objective memory impairment, normal general cognitive function, intact activities of daily living but no dementia. Mild cognitive impairment may also be referred to as incipient dementia, questionable dementia, age-associated cognitive decline and isolated memory impairment and the present invention embraces all these, and other commonly used, synonyms for mild cognitive impairment. Numerous studies have been performed on mild cognitive impairment and the reviews of these studies have indicated that individuals with mild cognitive impairment are at an increased risk of developing Alzheimer disease and, in most cases, convert to dementia and/or Alzheimer disease within several years.

In accordance with the present invention, there is provided the use of a 5-HT2C receptor antagonist in the manufacture of a medicament for the treatment of mild cognitive impairment, with the proviso that the 5-HT2C receptor antagonist is other than deramciclane or N-desmethylderamciclane or pharmaceutically acceptable acid addition salts thereof.

Compounds appropriate for use in the indications above will typically be 5-HT2C receptor antagonists. Receptor affinity may be known for individual compounds from the art, or may be determined either using the methods described herein or by alternative methods known from the art.

Any compound demonstrating 5-HT2C receptor antagonist activity may be used in the present invention. Suitable compounds include those described in the following patent publications: WO 97/16429, WO 97/44334, U.S. Pat. No. 5,010,078, EP 161,218, EP 401,707, EP 526,434, DE 02834114, EP 210,893, U.S. Pat. No. 3,580,916, U.S. Pat. No. 5,043,341, EP 620,222, EP 208,235, EP 437,790, DE 02614406, U.S. Pat. No. 4,338,317, EP 271,013, EP 110,435, EP 398,326, WO 92/05170, WO 95/01976, WO 96/23783, WO 98/04289, WO 97/48700, WO 00/48602, WO 00/26186, WO 99/58490, WO 99/52517, WO 99/51237, WO 99/46245, WO 99/43319, WO 99/33841, WO 99/33840, WO 99/25356, WO 99/09017, WO 99/03833, WO 99/00119, WO 98/56367, WO 98/52943, WO 98/50358, WO 98/50346, WO 98/50343, WO 98/41527, WO 98/38165, WO 98/30561, WO 98/30546, WO 98/24785, WO 98/21958, WO 98/04261, WO 97/48699, WO 97/41858, WO 97/39001, WO 97/37989, WO 97/20845, WO 97/12880, WO 97/08167, WO 97/06155, WO 97/00872, WO 96/39382, WO 96/30366, WO 96/24351, WO 96/23769, WO 96/18629, WO 96/14320, WO 96/11930, WO 96/11929, WO 96/02537, WO 95/29177, WO 95/25731, WO 95/24194, WO 95/21844, WO 95/18117, WO 95/12591, WO9 94/22871, WO 94/18958, WO 94/18182, WO 94/18170, WO 94/14801, WO 94/04533, WO 94/02462, WO 93/18028, WO 93/18026, WO 93/16081, WO 93/16051, WO 93/14758, WO 93/12790, WO 92/15302, WO 92/10192, WO 91/18602, WO 01/68585, WO 01/68067, WO 01/52855, WO 01/38329, WO 01/26621, WO 01/25229, WO 01/19371, WO 00/76984, WO 00/68181, WO 00/63185, WO 00/62782, WO 00/61129, WO 00/61128, WO 00/37068, WO 00/06165, U.S. Pat. No. 6,143,325, U.S. Pat. No. 5,854,248, U.S. Pat. No. 5,739,336, U.S. Pat. No. 5,693,645, U.S. Pat. No. 5,674,875, U.S. Pat. No. 5,498,618, U.S. Pat. No. 5,371,093, U.S. Pat. No. 5,266,571, U.S. Pat. No. 5,116,852, U.S. Pat. No. 5,106,855, U.S. Pat. No. 5,030,656, U.S. Pat. No. 5,013,735, U.S. Pat. No. 4,985,352, U.S. Pat. No. 4,914,107, U.S. Pat. No. 4,914,100, U.S. Pat. No. 4,906,639, U.S. Pat. No. 4,902,691, U.S. Pat. No. 4,891,376, U.S. Pat. No. 4,847,261, JP 13220375, JP 12204040, JP 11171865, JP 11080155, JP 10316634, JP 10077271, JP 09040646, JP 08053416, JP 08040999, JP 07228573, JP 07179337, JO 00158067, GB 02303303, GB 02301774, EP 01118610, EP 1070716, EP 01052245, EP 01000944, EP 00905136, EP 00797995, EP 00797994, EP 00769297, EP 00749971, EP 00749967, EP 00718299, EP 00700905, EP 00686393, EP 00682015, EP 0661266, EP 00657426, EP 006554440, EP 00613898, EP 00596449, EP 00559569, EP 00545120, EP 00522226, EP 00511074, EP 00511073, EP 00493687, EP 00484988, EP 00465398, EP 00452074, EP 00389352, EP 00388081, EP 00384228, EP 00379308, EP 00378468, EP 00375297, EP 00374042, EP 00373998, EP 00363963, EP 00354030, EP 00337136, EP 00332528, EP 00320983, EP 00218433 and EP 00145494.

The present invention thus includes the use of a compound as described in any of the above patent applications in the manufacture of a medicament for the treatment of cognitive dysfunction in or negative symptoms of schizophrenia, refractory schizophrenia, suicidality or mild cognitive impairment.

In addition to the compounds described in the above applications, the following compounds are suitable for use in the present invention: AHR-16303B (AH Robins Co. Inc), AP-792 and AT-1015 (Ajinomoto Co. Inc.), BMS-181102 (Bristol Myers Squibb), CV-5197 (Takeda Chemical Industries Ltd), dotarizine (Ferrer Internacional SA), E-2101 (Eisai Co Ltd), eltoprazine (Solvay SA), emopamil (Knoll AG), HT-90B (Chugai Pharmaceutical Co Ltd), ICI-169369 and ICI-170809 (Zeneca Group plc), LU-26042 and LU-29066 (H Lundbeck A/S), NPC-18166 (Scios Inc), Org-38457 (NV Organon), pelanserin (Cinvestav), perbufylline (Siegfried Group), SB-206553 and SB-242084 (SmithKline Beecham), SR-46615A (Sanofi Recherche SA), SUN-9221 (Suntory Ltd) tropoxin (Russian Academy Medical Science) and YM-992 (Yamanouchi Pharmaceutical Co Ltd).

The present invention thus also provides the use of any of AHR-16303B (AH Robins Co. Inc), AP-792 and AT-1015 (Ajinomoto Co. Inc.), BMS-181102 (Bristol Myers Squibb), CV-5197 (Takeda Chemical Industries Ltd), dotarizine (Ferrer Internacional SA), E-2101 (Eisai Co Ltd), eltoprazine (Solvay SA), emopamil (Knoll AG), HT-90B (Chugai Pharmaceutical Co Ltd), ICI-169369 and ICI-170809 (Zeneca Group plc), LU-26042 and LU-29066 (H Lundbeck A/S), NPC-18166 (Scios Inc), Org-38457 (NV Organon), pelanserin (Cinvestav), perbufylline (Siegfried Group), SB-206553 and SB-242084 (SmithKline Beecham), SR-46615A (Sanofi Recherche SA), SUN-9221 (Suntory Ltd) tropoxin (Russian Academy Medical Science) and YM-992 (Yamanouchi Pharmaceutical Co Ltd) in the manufacture of a medicament for use in the treatment of cognitive dysfunction in or negative symptoms of schizophrenia, refractory schizophrenia, suicidality or mild cognitive impairment.

Particularly preferred 5-HT2C receptor antagonists for the uses of the present invention include Ro-60-0759, RS-102221, SDZ-SER-082, Amersergide, ICI-169369, Sergolexole, Deramciclane, N-desmethyl-deramciclane, CGS-18102A and LU-26042. These compounds, together with methods for their preparation are described in WO 98/30546, U.S. Pat. No. 5,739,336, EP 473,550, U.S. Pat. No. 4,931,447, U.S. Pat. No. 4,435,405, U.S. Pat. No. 4,714,704, U.S. Pat. No. 4,342,762, U.S. Pat. No. 6,093,747, EP 161,218 and WO 93/14758 respectively.

Examples of candidate compounds for which the relative 5-HT2C affinity has been calculated according to formula I, above, from known 5-HT2C/5-HT2A/D2 affinities include those listed in Table 3, below, in which known anti-psychotic drugs are shown in bold typeface.

TABLE 3

| | | | | Formula I | | |
|---|---|---|---|---|---|---|
| | 5-HT2C | 5-HT2A | D2 | Affinity | | |
| Compound | Affinity | Affinity | Affinity | Ratio | Patent Number | CAS Number |
| Ro-60-0759 | 8.5 | 6.1 | 6 | 2.81 | WO-09830546 | |
| RS-102221 | 8.2 | 5.8 | 6 | 2.78 | US-05739336 | 185376-97-0 |
| SDZ-SER-082 | 7.8 | 6 | 6 | 2.60 | EP-0473550 | 141474-54-6 |
| Amesergide | 8.2 | 7.8 | 6 | 2.42 | US-04931447 | |
| ICI-169369 | 8 | 7.7 | 6.5 | 2.27 | US-04435405 | 85273-96-7 |
| Sergolexole | 7.2 | 7.2 | 6 | 2.20 | US-04714704 | |
| N-Desmethyl- | 7.2 | 7 | 7 | 2.20 | US06093747 | |
| deramciclane | | | | | | |
| Ritanserin | 9 | 9.6 | 7.4 | 2.15 | | |
| Clozapine | 8 | 8.2 | 7 | 2.12 | | |
| Deramciclane | 7.8 | 8 | 7 | 2.09 | US-04342762 | 120444-71-5 |
| CGS-18102A | 7.2 | 6.6 | 7.4 | 2.06 | EP-00161218 | |
| LU-26042 | 7.8 | 8.8 | 6.9 | 2.02 | WO-09312790/ | |
| | | | | | WO-09314758 | |
| Sertindole | 8.8 | 10 | | 1.99 | | |
| Olanzapine | 7.8 | 8.8 | | 1.93 | | |
| Ziprasidone | 7.9 | 8.9 | | 1.83 | | |

* Affinities expressed as pKi or pKd values

As is evident from this table, the relative 5-HT2C affinity is above 1.80 for each of the compounds listed and each of these compounds (excluding ritanserin, clozapine, sertindole, olanzapine and ziprasidone) is therefore suitable for the use of the present invention. In addition, ritanserin, sertindole, olanzapine and ziprasidone are suitable for use in the treatment of suicidality or mild cognitive impairment and clozapine is suitable for use in the treatment of mild cognitive impairment and suicidality in a non-schizophrenic patient.

In a further aspect, therefore, the present invention provides one of Ro-60-0759, RS-102221, SDZ-SER-082, Amesergide, ICI-169369, Sergolexole, CGS-18102A and LU-26042 for use in the manufacture of a medicament for the treatment of cognitive dysfunction in and/or negative symptoms of schizophrenia, refractory schizophrenia, suicidality or mild cognitive impairment.

In an alternative embodiment of this aspect of the invention, there is provided the use of deramiclcane, N-desmethyl-deramciclane or a pharmaceutically acceptable acid addition salt thereof in the manufacture of a medicament for the treatment of negative symptoms of schizophrenia (when not associated with cognitive dysfunction), refractory schizophrenia or suicidality.

Most preferred in this aspect of the invention is one of Amesergide, Sergolexole, CGS-18102A or LU-26042 for use in the manufacture of a medicament for the treatment of cognitive dysfunction in and/or negative symptoms of schizophrenia, refractory schizophrenia, suicidality or mild cognitive impairment; or deramiclcane, N-desmethylderamciclane or a pharmaceutically acceptable acid addition salt thereof in the manufacture of a medicament for the treatment of negative symptoms of schizophrenia (when not associated with cognitive dysfunction), refractory schizophrenia or suicidality.

1. Deramciclane

Deramciclane is (1R,2S,4R)-(−)-2-[2-(N,N-dimethylamino)-ethoxy]-2-phenyl-1,7,7-trimethylbicyclo[2.2.1]heptane and is described in U.S. Pat. No. 4,342,762.

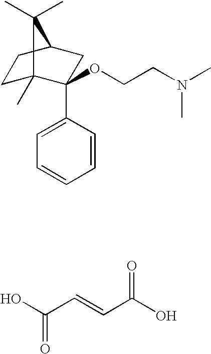

Deramciclane has the structural formula shown above. In man, deramciclane undergoes biotransformation into N-desmethyl-deramciclane, which is an active metabolite with even more pronounced 5-HT2C receptor antagonism (U.S. Pat. No. 6,093,747).

2. Amesergide

Amesergide is N-cyclohexyl-1-isopropyl-6-methyl-ergoline-8-carboxamide and has the following structural formula:

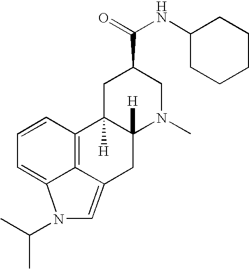

The compound is described in U.S. Pat. No. 4,931,447 and is known to block 5HT2A/2C mediated elevations of prolactin levels induced by D-fenfluramine. The metabolism of amesergide has been evaluated in rhesus monkeys, with maximum values of parent compounds or metabolites (4-hydroxy and desisopropyl species) usually occurring on day 35, with minimum values occurring on day 365, suggesting that elimination or transformation of amesergide was enhanced as the study progressed.

3. Sergolexole

Sergolexole (chemical name: [trans(8β)]6-methyl-1-[1-methylethyl]ergoline-8-carboxylic acid, 4-methoxycyclohexyl ester (maleate salt)) and its preparation, is described in U.S. Pat. No. 4,714,704. The compound, having the structural formula shown below, is known to be useful in the treatment of migraine. 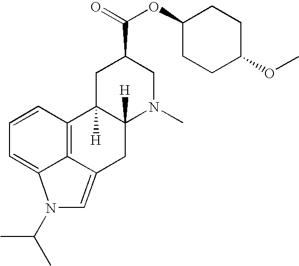
4. CGS-18102A

This compound (10-methoxy-4-propyl-1,2,3,4a,5,10b-hexahydro-4H-[1]-benzopyrano[3,4-b]pyridine hydrochloride) is described in EP 161,218 and has the following structural formula:

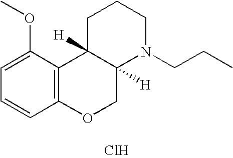

This compound is a potent 5HT2A/2C receptor antagonist that has been proposed for use as an anxiolytic or antidepressant drug.

5. LU-26042

This compound 1-{2-[4[(2,5-dimethyl-3-(4-fluorophenyl)-1H-indol-1-yl)-1-piperidinyl]ethyl}-2-imidazolidinone, is a known 5-HT2A/2C receptor antagonist, described in WO 93/12970 and WO 93/14758. The compound has the following structural formula:

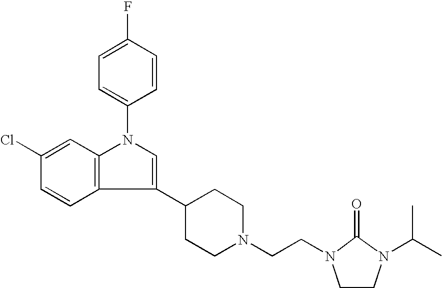

In addition to the above, the present invention also provides a product containing a 5-HT2C receptor antagonist and a typical antipsychotic as a combined preparation for simultaneous, separate or sequential use in schizophrenia therapy. In a preparation suitable for this use, the 5-HT2C receptor antagonist is as described herein. The typical antipsychotic would generally be a compound suitable for the general treatment of schizophrenia, and preferably suitable for those sub-classes of schizophrenia not treatable by a 5-HT2C receptor antagonist, for example the positive symptoms. An example of such a compound includes haloperidol, chlorpromazine, fluphenazine, fluspirilene, loxapine, pimozide, sulpiride, thioridazine and thiothixene. The combination of the two compounds would therefore result in the treatment of all or substantially all schizophrenic symptoms.

Analysis of “Atypical” Antipsychotics

With the recent introduction of several “atypical” antipsychotic drugs to the market, a large amount of information on clinical efficacy and side effects of these drugs has become available. Although few of these recently introduced drugs have been compared in clinical trials side by side, recent published reports provide much greater insight into possible relationships between clinical effects and pharmacological properties of the drugs. Thus, the clinical characteristics of the drugs clozapine, olanzapine, risperidone, seroquel (quetiapine), sertindole and ziprasidone were compared and rated based on recent literature reports (Meltzer 2000, Javitt 2001, Azorin et al. 2001, Sauriol et al. 2001, Conley and Mahmoud 2001, Chakos et al. 2001, Cuesta et al. 2001, Wetterling 2001, Taylor and McAskill 2000, Purdon et al. 2000, Leucht et al. 1999, Pezawas et al. 2000, Lewis et al. 2000, Meltzer and McGurk 1999, Arvanitis and Miller, 1997 and Tran et al. 1997).

The different clinical read-outs have been grouped: 1) extra-pyramidal side effects (includes causation of tardive dyskinesia) 2) improvement of negative symptoms (includes effects on flatness, social withdrawal and anhedonia) 3) improvement of cognitive dysfunction 4) increases in serum prolactin levels 5) increase in weight gain. These effects, in addition to suppressing positive symptoms of the disease, represent features desirable for candidate compounds.

Tables 4 and 4A show an overview of clinical characteristics (A) and certain receptor affinity ratios (B) of currently marketed and clinically well evaluated “atypical” antipsychotic drugs*

TABLE 4A

| | Extra- | Improve- | Improve- | | |
|---|---|---|---|---|---|
| “Atypical” | pyramidal | ment | ment | Increases | |
| Anti- | side | negative | cognitive | prolactin | Weight |
| psychotic | effects | symptoms | deficits** | levels | Gain |
| Clozapine | 3 | 1 | 1-2 | 3 | 1 |
| Risperidone | 1 | 3 | 2-3 | 1 | 2-3 |
| Olanzapine | 1-2 | 2 | 1-2 | 2 | 2 |
| Sertindole | 3 | 2 | | 3 | 2-3 |
| Seroquel | 2 | 3 | 2-3 | 3 | 2 |
| Ziprasidone | 2 | 2 | | 3 | 3 |

*grading: 1-high, 2-medium and 3-low probability.
References used: (Meltzer 2000, Javitt 2001, Azorin et al. 2001, Sauriol et al. 2001, Conley and Mahmoud 2001, Chakos et al. 2001, Cuesta et al. 2001, Wetterling 2001, Taylor and McAskill 2000, Leucht et al. 1999, Pezawas et al. 2000, Lewis et al. 2000, Arvanitis and Miller, 1997 and Tran et al. 1997)
**for ratings of specific cognitive measures see Table 3.

TABLE 4B

| | | 5-HT2C/ | | | |
|---|---|---|---|---|---|
| “Atypical” | | 5-HT2A | 5-HT2C/ | | |
| Anti- | 5-HT2A/ | ratio | D2 ratio | (2C/2A) + | H1/D2 |
| psychotic | D2 ratio | (2C/2A) | (2C/D2) | (2C/D2) | ratio |
| Clozapine | 1.22 | 0.98 | 1.14 | 2.12 | 1.23 |
| Risperidone | 1.09 | 0.79 | 0.90 | 1.69 | 0.9 |
| Olanzapine | 1.13 | 0.88 | 1.04 | 1.92 | 1.05 |
| Sertindole | 1.25 | 0.89 | 1.02 | 1.91 | 0.71 |
| Seroquel | 1.15 | 0.81 | 0.92 | 1.73 | 1.08 |
| Ziprasidone | 1.06 | 0.89 | 0.94 | 1.83 | 0.87 |

The clinical characteristics of antipsychotic drugs can be correlated with their pharmacological properties.

1. Extra Pyramidal Side Effects (EPS)

Although the induction of EPS has been an important part of “atypicality” as defined by Meltzer et al. (1989), the current analysis is the first to demonstrate that there is a correlation between the level of induction of EPS and the 5-HT2A/D2 receptor affinity ratio (FIG. 1, Panel A). As many 5-HT2A receptor antagonists also interact with 5-HT2C receptors, it was investigated whether an association existed between EPS and relative 5-HT2C receptor affinity (expressed as the sum of the 5-HT2C/5-HT2A and 5-HT2C/D2 affinity ratios in formula I above). FIG. 1 (Panel C) demonstrates the absence of a robust relationship. Further possible correlations between receptor affinity ratios and the level of EPS induction were assessed and as shown in FIG. 1 (Panels E, G, I, K, M, O, Q). Although the correlation between EPS liability and 5-HT2A/D2 ratio is not perfect (ziprasidone appears to be an outlier) it is possible that over time, when head to head comparative trials have been done and drugs have been prescribed more widely, the correlation will improve.

In summary it is suggested that blockade of the 5-HT2A receptor is an important factor in the suppression (or prevention) of D2 receptor-induced EPS.

2. Improvement of Negative Symptoms

Thus far, there is no (new) antipsychotic which appears to have comparable efficacy to clozapine in treating negative symptoms. Similarly, clozapine is the most effective drug to treat neuroleptic-refractory patients. The present analysis demonstrates that of all pharmacological properties possessed by the novel antipsychotic agents, the relative 5-HT2C receptor affinity (expressed as the sum of 5-HT2C/5-HT2A and 5-HT2C/D2 affinity ratios in the formula above) was most discernibly correlated with improvement of negative symptoms (see FIG. 1, Panel B). Similarly, the 5-HT2C/5-HT2A and 5-HT2C/D2 affinity ratios separately demonstrated clear correlations with efficacy to treat negative symptoms (FIG. 1, Panels F and H).

Thus, although it is not suggested that the absolute 5-HT2C receptor affinity (see Table 1) correlates with this clinical effect (as previously investigated by Roth et al. 1992), it is suggested that the relative 5-HT2C receptor affinity (when compared to both 5-HT2A and D2 receptor affinities) shows a clear relationship with therapeutic efficacy in the treatment of negative symptoms. Comparisons of other pharmacological properties of the antipsychotic drugs did not reveal the same degree of correlation (FIG. 1, Panels D, J, L, N, P and R). For example, a correlation between 5-HT2A/D2 affinity ratio and improvement of negative symptoms was absent (FIG. 1, Panel D).

In summary, it is suggested that blockade of the 5-HT2C receptor is important in treating cognitive dysfunction in or the negative symptoms of schizophrenia, refractory schizophrenia, suicidality or mild cognitive impairment.

[image]

FIG. 2. Correlation Plots of Clinical Read-Outs for Atypical Antipsychotic Drugs and Neurotransmitter Receptor Affinity Ratios (See Table 4a and b for data)

3. Improvement of Cognitive Deficits

There is limited information on the effects of novel antipsychotic drugs on cognitive deficits of schizophrenic patients. It is evident, however, that conventional neuroleptic drugs do not improve cognitive dysfunction in schizophrenia. Reportedly, clozapine has significant effects on several cognitive functions as determined by a range of neuropsychological tests (Hagger et al. 1993, Green et al. 1997). Similarly, preliminary studies have demonstrated improvement to cognitive function by olanzapine and to a lesser extent by risperidone and quetiapine (Meltzer 2000b-chapter int, Meltzer and McGurk 1999, Cuesta et al. 2001, Purdon et al. 2000, Velligan et al. 2002, Purdon et al. 2001, Meltzer 2000). Table 5 represents the different levels of effect of the drugs as reported in these literature sources.

TABLE 5
Effects of “Atypical” Antipsychotic Drugs on Various
Cognitive Functions

| Cognitive Effect | Clozapine | Olanzapine | Risperidone | Quetiapine |
|---|---|---|---|---|
| Attention | ++ | − | + | + |
| Executive Function | + | + | + | + |
| Verbal Learning | +/− | ++ | − | − |
| Verbal Memory | + | ++ | − | + |
| Working Memory | +/− | − | ++ | − |
| Verbal Fluency | ++ | ++ | − | + |

Although the trials have been sparse, it appears that clozapine and olanzapine may be more effective in improving cognitive deficits in schizophrenia than risperidone or quetiapine. As clinical data on only four novel antipsychotics was available, correlations between receptor affinity and clinical effect are weak (see FIG. 2). However, when compared to other correlations, it seems that the association with relative 5-HT2C receptor affinity is most relevant (Panel A). Although, the relationship with 5-HT2A or 5-HT6 cannot be excluded (Panels B and D), the 5-HT2C receptor affinity appears to be a contributing factor when compared to the 5-HT2A affinity of the drugs (Panel C).

4. Increases in Serum Prolactin Levels

As has been reported previously, there does not seem to be a clear correlation between pharmacological characteristics and the level of serum prolactin increase of antopsychotic agents. As pictured in FIG. 2 (Panel A) a rather unclear trend may be observed of relative affinity to the D2 receptor. We do not wish to speculate however on the possible pharmacological properties involved in the response.

5. Weight Gain

Increase in weight gain, varying from 0.8-3.5 kg per month, has been reported for a number of novel antipsychotic agents. Haloperidol in contrast does not have such an effect. Clozapine, olanzapine and seroquel reportedly do cause weight gain (Taylor and McAskill, 2000, Wetterling 2001). As demonstrated in FIG. 2 (Panel B) a relationship between H1/D2 receptor affinity and increasing liability for weight gain exists. This is not a novel finding and has been previously reported (Wirshing et al. 1999).

To conclude, rather than evaluating the “atypicality” of antipsychotic drugs the present results demonstrate that different pharmacological properties of antipsychotic agents may be responsible for different features of atypicality of these drugs. There is likely to be a significant grey area whereby antipsychotics possess certain atypical characteristics but not all.

Importantly, new correlations between the 5-HT2A/D2 receptor affinity ratio and causation of EPS but more importantly between relative 5-HT2C receptor affinity and the improvement of negative symptoms, cognitive deficits, refractory schizophrenia, suicidality or mild cognitive impairment have been revealed. This finding will be of great value in the development of drugs that are effective in the treatment of patients suffering from these symptoms.

5-HT2C Receptor Antagonism and Suicide

As mentioned above Niswender and colleagues (2001) have reported increased levels of 5-HT2C messenger RNA editing. It remains unclear, however, what role the 5-HT2C receptor would play and whether agonists or antagonists to the 5-HT2C receptor could have beneficial effects on suicidal behaviour. Further analysis of the data published by Niswender et al. (2001) demonstrates that there may be differences between male and female patients, their drug treatments, levels of 5-HT2C receptor mRNA editing and possibly suicide rate (see Table 6).

TABLE 6
Levels of 5-HT2C receptor mRNA editing (A form) in
depressive, schizophrenic and control subjects (with and
without suicide)

| | Control | Major Depression* | Schizophrenia* |
|---|---|---|---|
| Females | | | |
| | 82.8 | 83.4 | 77.6 |
| | 83.2 | 86.6 | 78.6 |
| | 84.2 | 87.8 | 79 |
| | | 90.3 | [symbol] |
| | | | 87.8 |
| | | | 89 |
| Average | 83.4 | 87.0 | 82.1 |
| SEM | 0.51 | 1.65 | 2.23 |
| Males | | | |
| | 77.3 | 77.8 | 73.6 |
| | 79.9 | 80.8 | 82.7 |
| | 82.2 | 80.8 | 84.4 |
| | 82.9 | 84.3 | [symbol] |
| | 83.1 | 84.8 | 85.9 |
| | 84.5 | 84.8 | 86.2 |
| | 84.7 | 87.3 | 86.6 |
| | 85.1 | 87.5 | |
| | 85.5 | 88.6 | |
| | 90.5 | | |
| Average | 83.6 | 84.1 | 83.5 |
| SEM | 1.17 | 1.28 | 1.85 |

*in bold: subject comitted suicide/in italics subject's latest drug treatment blocked 5-HT2C receptors

A strong trend for women with major depression to have 40 increased levels of the Edit-A form of 5-HT2C receptor mRNA was observed (p=0.04, Student's t-test, p=0.09, ANOVA). Also, the data were re-analysed considering the type of drug treatment the donor received (the latest before death). The types of drug treatment were classified as 1) drugs known to antagonise the 5-HT2C receptor (such as clozapine, loxapine or olanzapine) and 2) other drugs (e.g. haloperidol) or no drug treatment at all. Then, a trend for a correlation between level of Edit-A 5-HT2C receptor mRNA and 5-HT2C receptor blocking drug treatments was revealed (see Table 7). Further, although the sample set was small, a possible relationship between treatment with 5-HT2C receptor blocking drugs and a lower suicide rate was observed.

TABLE 7
Comparison of Edit-A 5-HT2C receptor mRNA levels in
schizophrenic patients with and without drug treatment*
blocking 5-HT2C receptors

Schizophrenia

| | Drug treatment with | Drug treatment without |
|---|---|---|
| | 5-HT2C antagonism | 5-HT2C antagonism |
| | 78.6 | 77.6 |
| | 80.7 | 79 |
| | 73.6 | 87.8 |
| | 82.7 | 89 |
| | 84.4 | 85.9 |
| | 84.8 | 86.2 |
| | | 86.6 |
| Average | 80.8 | 84.6 |
| SEM | 1.89 | 1.81 |
| p (t-test) = | 0.07 | |
| p (ANOVA) = | 0.14 | |

*The subject's latest drug treatment included blockade of 5-HT2C receptors

Thus, of seven schizophrenic patients not treated with drugs blocking the 5-HT2C receptor, four committed suicide, whereas of the six patients previously treated with 5-HT2C receptor blocking antipsychotic drugs, only two committed suicide. Larger studies will be needed to confirm the present observations. Nevertheless, it may be possible that the 5 HT2C blocking effects of antipsychotic drugs reduce suicide completion rates in schizophrenic, and perhaps other, psychiatric patients.

Thus, to conclude, the present study is the first to reveal a distinct role for 5-HT2C receptor antagonists in the treatment of the negative symptoms or cognitive deficits of schizophrenia or refractory schizophrenia.

Further, 5-HT2C receptor antagonists may be of benefit in the treatment of suicidality or mild cognitive impairment.

While it is possible for the 5-HT2C receptor antagonist to be administered alone, it is preferable to present the compound as a pharmaceutical composition (e.g. formulation) comprising at least one active compound together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, ointments, gels, pastes, creams, sprays, mists, foams, lotions, oils, suppositories, boluses or sustained release formulations.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

A more recently devised approach for parenteral administration employs the implantation of a slow-release or sustained-release system, such that a constant level of dosage is maintained. See, e.g., U.S. Pat. No. 3,710,795.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Depot formulations, such as those comprising a microsphere-based delivery system wherein the active compound is incorporated into a matrix of poly-(DL-lactide-co-glycolide) (PLG), may otherwise be used. In such instances, release profiles can be adjusted by manipulation of formulation parameters and through control of the fabrication process.

Formulations suitable for topical administration may comprise a patch or a dressing such as a bandage or adhesive plaster impregnated with active compounds and optionally one or more excipients or diluents.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of a 5-HT2C receptor antagonist will be similar to that described during the original preparation and use of the compound. This may be in the form of a single bolus dose or more preferably in multiple applications or a sustained release preparation. Factors such as age, weight, sex and presence or absence of other diseases, may have a bearing on the suitable daily dose.

When the 5-HT2C receptor antagonist is deramciclane or N-desmethyl-deramciclane, an oral daily dose of between 10 mg and 60 mg will be appropriate, preferably of about 30 mg.

When the 5-HT2C receptor antagonist is Amesergide, an oral daily dose of between 50 mg and 150 mg will be appropriate, preferably of about 100 mg.

When the 5-HT2C receptor antagonist is Sergolexole, an oral daily dose of between 10 mg and 150 mg will be appropriate, preferably of about 50 mg.

The present invention furthermore provides a product, for example a kit, containing a 5HT2C receptor antagonist together with a typical antipsychotic as a combined preparation for simultaneous, separate or sequential use in schizophrenia or suicidality therapy or the treatment of cognitive impairment.

In this aspect of the invention, the 5HT2C receptor antagonist is substantially as herinabove defined, or may be identified by use of a method substantially as hereinabove defined. Typical antipsychotics are known and available. The choice of antipsychotic will depend on various factors such as, for example, the nature and severity of the condition to be treated, as well as the particular 5HT2C receptor antagonist also forming a part of the product.

The precise format for performing methods of the invention may be varied by those of skill in the art using routine skill and knowledge.

Of course, the person skilled in the art will design any appropriate control experiments with which to compare results obtained in methods of the invention.

All documents mentioned in this specification are hereby incorporated by reference.

### EXAMPLES

This exemplification details the determination of affinity values (affinity values expressed as Ki or Kd or antagonist activity as IC50, Kb or A2- or the -log of any of these values) of compounds at either recombinant receptors expressed stably in a cell line (example 1) or transiently (example. 2). Furthermore references are given for the determination of such values in tissue homogenates from rat, mouse, human and porcine brain using a variety of methods.

### Example 1

### Method Taken from Berg et al. 1999

Cell Culture. Chinese hamster ovary K1 (CHO-K1) cell lines that stably express human 5-HT2C receptors at '250 fmol/mg (“low” expressing, CHO-1C19) and 5 to 10 pmol/mg (“high” expressing, CHO-1C7) were used in this study. Cells were maintained in a minimal essential medium supplemented with 5% fetal bovine serum and 300 mg/ml hygromycin. For all experiments, cells were seeded into 12- or 24-well tissue culture vessels at a density of 4 3 10 4 cells/cm². After a 24-h plating period, cells were washed with Hanks' balanced salt solution (HBSS) and placed into Dulbecco's modified Eagle's medium/F-12 [1:1] with 5 mg/ml insulin, 5 mg/ml transferrin, 30 nM selenium, 20 nM progesterone, and 100 mM putrescine (serum free media) and grown for an additional 24 h before experimentation. The absence of receptor reserve for 5-HT on both effector pathways (PLC and PLA2) coupled to the human 5-HT2C receptor in CHO-1C19 cells (Berg et al., 1998) has been previously demonstrated.

IP Accumulation and AA Release Measurements.

IP accumulation and AA release were measured as described previously (Berg et al., 1994a, 1996, 1998). Unless stated otherwise, measurements of PLC-mediated IP accumulation were made from the same multiwell (simultaneously) as PLA2-AA release measurements (Berg et al., 1998). Briefly, cells in serum-free medium were labeled with 1 mCi/ml [3H]-myoinositol (25 Ci/mmol) for 24 h and with 0.1 mCi/ml [3H]AA (220 Ci/mmol) for 4 h at 37° C. After the labeling period, cells were washed three times with HBSS containing calcium and magnesium, 20 mM HEPES, and 0.1% fatty acid-free bovine serumalbumin (BSA; experimental medium). Between washes, the cells were incubated for 5 min in a 37° C. water bath (15-min total wash and preincubation time). After the wash procedure, cells were incubated in 0.5 ml of experimental medium containing vehicle (H20 or 0.01% DMSO) or the indicated drug concentrations. For measurement of basal effector activity, cells were incubated at 37° C. for 25 min. For measurement of agonist-mediated stimulation of effector activity, cells were incubated at 37° C. for 10 min. After incubation, aliquots (100 ml) of cell media were added directly to scintillation vials for measurement of [3H] content (Berg et al., 1996, 1998). The remaining media were aspirated quickly and 1 ml 10 mM formic acid (4° C.) was added to extract the accumulated [3H]-IPs (IP1, IP2, and IP3, collectively referred to as IP; Berg et al., 1994a). For some experiments, data were normalized to protein content, which was measured according to the method of Lowry et al. (1951).

Receptor Binding Studies.

5-HT2C receptor saturation binding experiments were done as described previously (Berg et al., 1994a). Briefly, cells were washed twice with HBSS, scraped, and centrifuged at 500 g for 5 min. Cell pellets were flash frozen in liquid nitrogen and stored at 2135° C. until use. All membrane preparation procedures were done at 4° C. Cell pellets were thawed, resuspended in 20 volumes of homogenization buffer (50 mM HEPES, 2.5 mM MgCl₂, 2.0 mM EGTA pH 7.4 at 22° C.), homogenized twice with a polytron (setting no. 7) for 15s (separated by 15s), and centrifuged (39,000 g; 4° C.; 10 min). The resulting membrane pellet was washed three times with homogenization buffer and resuspended in assay buffer (homogenization buffer containing 0.1% ascorbic acid) for use in the binding assay. Aliquots (250 ml) of membrane suspension (50 mg protein) were incubated (60 min; 37° C.; total volume 5 500 ml) with 13 concentrations (0.01-40 nM) of [3H]-mesulergine. Nonspecific binding was determined in the presence of 1 mM mianserin. Samples were filtered through polyethyleneimine-coated Whatman GF/C filters (Whatman Inc., Clifton, N.J.) with a Brandel Cell Harvester (Brandel Laboratories, Gaithersburg, Md.). The filters were washed twice with 1.5 ml ice-cold buffer and counted with a Beckman LS7500 liquid scintillation counter (Beckman Instruments, Berkeley, Calif.). Protein was determined with the method of Lowry et al. (1951) using BSA as a standard.

Data Analysis

Concentration response data were fit with non-linear regression to the model:

$$E = \frac{{E}_{max}}{1 + {[ \frac{{EC}_{50}}{A} ]}^{n}} \qquad ( 1 )$$

where E is the measured response at a given agonist concentration (A), Eₘₐₓ=maximal response, EC₅₀=the concentration of agonist producing half-maximal response, and n=slope index. Calculation of apparent antagonist dissociation constants (K_{B}) was determined with the equation:

$${K}_{B} = \frac{[ B ]}{dr - 1} \qquad ( 2 )$$

where B is the concentration of the antagonist used and dr represents the ratio (dose ratio) of concentrations (EC₅₀) that produced equivalent responses in the absence and presence of antagonist. Data from saturation binding studies were analyzed with nonlinear regression analysis. After fitting nonspecific data to the equation describing a straight line with the origin at 0,0 (y=mx) to determine m, total binding data were fit to equation 3 to provide estimates of Bₘₐₓ, K_{d}, and slope factor (n):

$$B = \frac{{B}_{max}}{{[ \frac{{K}_{d}}{[ A ]} + 1 ]}^{n}} + m [ A ] \qquad ( 3 )$$

where m is the slope of the linear regression line for nonspecific binding.

### Example 2

### Method Taken from Herrick-Davis et al. 2000

Cell Culture and Transfection

COS-7 cells were grown in Dulbecco's modified Eagle's medium (DMEM) with 10% fetal bovine serum in a humidified incubator with 5% CO₂ at 37° C. Twenty-four hours before transfection, cells were seeded at 10⁵ cells/well in 24-well cluster plates for IP assays and for [³H]mesulergine binding studies performed in parallel to monitor receptor expression. Cells were transfected with the rat or human 5-HT2C receptor by combining 2 ml of lipofectAMINE with 0.5 mg of plasmid DNA in 400 ml of serum-free DMEM and added to each well for 5 h at 37° C./5% CO₂. For radioligand binding studies, COS-7 cells were seeded at 80% confluence in 100-mm dishes and transfected with 5 mg of plasmid DNA, 20 ml of lipofectamine in 4 ml of serum-free DMEM for 5 h at 37° C./5% CO₂. After transfection, cells were returned to complete culture medium for 48 h before membrane preparation for radioligand binding studies.

IP Production Assays

IP production was measured according to the method of Herrick-Davis et al., 1999. In brief, 24 h after transfection COS-7 cells were washed with PBS and labeled over-night with 0.5 mCi/well of myo-[³H]inositol in inositol-free/serum-free DMEM at 37° C./5% CO₂. After labeling, cells were washed with PBS and preincubated in inositol-free/serum-free DMEM with 10 mM LiCl and 10 mM pargyline (assay medium) for 10 min. Antipsychotic drugs were added during the 10-min preincubation. 5-HT, or assay medium alone, was added to each well and incubation continued for an additional 35 min to determine basal activity. Assay medium was removed and cells were lysed in 200 ml of stop solution (1 M KOH/18 mM sodium borate/3.8 mM EDTA) and neutralized by adding 200 ml of 7.5% HCl. The contents of each well were extracted with 3 volumes of chloroform:methanol (1:2, v/v) and centrifuged 5 min at 10,000 g, and the upper layer was loaded onto a 1-ml AG1-X8 resin (100-200 mesh) column. Columns were washed with 10 ml of 5 mM myo-inositol and 10 ml of 5 mM sodium borate/60 mM sodium formate. Total [³H]IPs were eluted with 3 ml of 0.1 M formic acid/1 M ammonium formate. Radioactivity was measured by liquid scintillation counting in Ecoscint cocktail.

Radioligand Binding

Membranes were prepared by scraping a confluent 100-mm dish of transfected COS-7 cells into 20 ml of 50 mM Tris-HCl/5 mM MgSO₄/0.5 mM EDTA, pH 7.4 (assay buffer) and centrifugation at 10,000 g for 30 min. Membranes were resuspended in 20 ml of assay buffer, homogenized, and centrifuged again. After resuspension in 15 ml of assay buffer, 0.5-ml membrane aliquots were added to each assay tube containing 1 nM [³H]mesulergine and varying concentrations of competing drug in a final volume of 1 ml. Mianserin (10 mM) was used to define nonspecific binding. Samples were incubated at 37° C. for 30 min, filtered through glass fiber filters (presoaked in 0.3% polyethylenamine) on a Brandel cell harvester, and counted in Ecoscint cocktail in a liquid scintillation counter (Beckman, Berkeley, Calif.) at 40% efficiency.

Data Analyses. Data analyses were performed using Prism software (GraphPad, San Diego, Calif.). The Cheng/Prusoff equation was used to calculate Ki values from IC50 values.

Further examples are as detailed in the method sections of the following references:

The binding of serotonergic ligands to the porcine choroids plexus: characterization of a new type of serotonin recognition site” Pazos A, Hoyer D and J M Palacios, Eur J. Pharmacol. 1985, 106:539-546

Molecular pharmacology of 5-HT1 and 5-HT2 recognition sites in rat and pig brain membranes: radioligand binding studies with [3H)5-HT, (3H]8-OH-DPAT, (−)[¹²⁵I]iodocyanopindolol, [3H]mesulergine and [3H]ketanserin” Hoyer D, Engel G and HO Kalkman, Eur J. Pharmacol. 1985, 118(1-2):13-23

“Quantitative autoradiographic mapping of serotonin receptors in the rat brain. I. Serotonin-1 receptors” Pazos A and J M Palacios, Brain Res. 1985; 346(2):205-30

“Serotonin 5-HT1C receptors are expressed at high density on choroid plexus tumors from transgenic mice” Yagaloff K A, Lozano G, Van Dyke T, Levine A and P R Hartig, Brain Res. 1986, 385(2):389-94

“[125I]LSD labels 5-HT1C recognition sites in pig choroid plexus membranes. Comparison with [3H]mesulergine and [3H]5-HT binding”. Hoyer D, Srivatsa S, Pazos A, Engel G and J M Palacios, Neurosci Lett. 1986, 69(3):269-74

“Serotonin receptors in the human brain. I. Characterization and autoradiographic localization of 5-HT1A recognition sites. Apparent absence of 5-HT1B recognition sites” Hoyer D, Pazos A, Probst A and J M Palacios, Brain Res. 1986, 376(1):85-96

“Serotonin receptors in the human brain. II. Characterization and autoradiographic localization of 5-HT1C and 5-HT2 recognition sites” Hoyer D, Pazos A, Probst A and J M Palacios, Brain Res. 1986, 376(1):97-107

“Serotonin receptors in the human brain—III. Autoradiographic mapping of serotonin-1 receptors” Pazos A, Probst A, and J M Palacios, Neuroscience. 1987, 21(1):97-122

### BIBLIOGRAPHY

Altar C A, Wasley A M, Neale F F et al, 1986, Typical and atypical antipsychotic occupancy of D-2 and S-2 receptors: An autoradiographic analysis in rat brain. Brain Res. Bull. 16: 517-525.
Andreasen N C, 1982, Negative symptoms in schizophrenia. Definition and reliability. Arch Gen Psychiatry; 39(7):784-8.
Arvanitis L A, Miller B G, 1997, Multiple fixed doses of “Seroquel” (quetiapine) in patients with acute exacerbation of schizophrenia: a comparison with haloperidol and placebo. The Seroquel Trial 13 Study Group. Biol Psychiatry. 42(4):233-46.
Azorin J M, Spiegel R, Remington G, Vanelle J M, Pere J J, Giguere M, Bourdeix, 2001, A double-blind comparative study of clozapine and risperidone in the management of severe chronic schizophrenia. Am J Psychiatry. 158(8):1305-13.
Baron M, 2001, Genetics of schizophrenia and the new millennium: progress and pitfalls. Am J Hum Genet. 68(2):299-312.
Bassett A S, Chow E W, Waterworth D M, Brzustowicz L, 2001, Genetic insights into schizophrenia. Can J Psychiatry. 46(2):131-7.
Berg K A, Clarke W P, Sailstad C, Saltzman A, Maayani S, 1994, Signal transduction differences between 5-hydroxytryptamine type 2A and type 2C receptor systems. Mol Pharmacol. 46(3):477-84.
Berg K A, Maayani S, Clarke W P, 1996, 5-hydroxytryptamine2C receptor activation inhibits 5-hydroxytryptamine1B-like receptor function via arachidonic acid metabolism. Mol Pharmacol. 50(4):1017-23.
Berg K A, Maayani S, Goldfarb J, Scaramellini C, Leff P, Clarke W P, 1998, Effector pathway-dependent relative efficacy at serotonin type 2A and 2C receptors: evidence for agonist-directed trafficking of receptor stimulus. Mol Pharmacol. 54(1):94-104.
Berg K A, Stout B D, Cropper J D, Maayani S, Clarke W P, 1999, Novel actions of inverse agonists on 5-HT2C receptor systems. Mol Pharmacol. May; 55(5):863-72.
Bolden C, Cusack B and Richelson E, 1992, Antagonism by antimuscarinic and neuroleptic compounds at the five cloned human muscarinic cholinergic receptors expressed in chinese hamster ovary cells. J. Pharmacol. Exp. Ther. 260: 576-580.
Bunney B G, Bunney W E and A Carlsson, 2000, Schizophrenia and Glutamate: An Update, Psychopharmacology—The Fourth Generation of Progress, The American College of Neuropsychopharmacology website: http://www.acnp.org/g4/4thgen.php
Bymaster F P, Calligaro D O, Falcone J F, et al, 1996, Radioreceptor binding profile of the atypical antipsychotic olanzapine. Neuropsychopharmacol. 14: 87-96.
Canton H, Verriele L, Colpaert F C, 1990, Binding of typical and atypical antipsychotics to 5-HT1C and 5-HT2 sites: clozapine potently interacts with 5-HT1C sites. Eur J. Pharmacol. 191(1):93-6.
Carlsson A, 1988, The current status of the dopamine hypothesis of schizophrenia. Neuropsychopharmacol. 1:179-186.
Carlsson A, Waters N, Holm-Waters S, Tedroff J, Nilsson M, Carlsson M L, 2001, Interactions between monoamines, glutamate, and GABA in schizophrenia: new evidence. Annu Rev Pharmacol Toxicol. 41:237-60.
Carlsson A, Waters N, Waters S, Carlsson M L, 2000, Network interactions in schizophrenia—therapeutic implications. Brain Res Brain Res Rev. 31(2-3):342-9.
Casey D E, 1994, Motor and mental aspects of acute extrapyramidal syndromes. Acta Psychiatr Scand Suppl.380:14-20.
Chakos M, Lieberman J, Hoffman E, Bradford D, Sheitman B, 2001, Effectiveness of second-generation antipsychotics in patients with treatment-resistant schizophrenia: a review and meta-analysis of randomised trials. Am J Psychiatry. 158(4):518-26.
Conley R R, Mahmoud R, 2001, A randomized double-blind study of risperidone and olanzapine in the treatment of schizophrenia or schizoaffective disorder. Am J Psychiatry 158(5):765-74.
Corbett R, Hartman H B, Kerman L L, et al, 1993, Effects of atypical antipsychotic agents on social behaviour in rodents. Pharmacol. Biochem. Beh. 45: 9-17.
Creese I, Burt D R and Snyder S H, 1976, Dopamine receptor binding predicts clinical and pharmacological potencies of anti-schizophrenic drugs. Science 192: 481-483.
Creese I, Burt D R, Snyder S H, 1976, Dopamine receptor binding predicts clinical and pharmacological potencies of antischizophrenic drugs. Science. 192(4238):481-3.
Cuesta M J, Peralta V, Zarzuela A, 2001, Effects of olanzapine and other antipsychotics on cognitive function in chronic schizophrenia: a longitudinal study. Schizophr Res. 48(1):17-28.
Dean B, 2000, Signal transmission, rather than reception, is the underlying neurochemical abnormality in schizophrenia. Aust N Z J Psychiatry. 34(4):560-9.
Deniker, P. (1983) Discovery of the clinical use of neuroleptics. In Discoveries in Pharmacology (eds M. J. Parnham & J. Bruinvels), Vol 1, pp. 163-180. Amsterdam: Elsevier Science Publishers.
Du L, Faludi G, Palkovits M, Bakish D, Hrdina P D, 2001, Serotonergic genes and suicidality. Crisis 22(2):54-60.
Farde L, Nordstrom A L, Wiesel F A, Pauli S, Halldin C, Sedvall G, 1992, Positron emission tomographic analysis of central D1 and D2 dopamine receptor occupancy in patients treated with classical neuroleptics and clozapine. Relation to extrapyramidal side effects. Arch Gen Psychiatry. 49(7):538-44.
Farde L, Wiesel F-A, Nordstrom A-L, et al, 1989, D₁ and D₂-dopamine receptor occupancy during treatment with conventional and atypical neuroleptics. Psychopharmacol. 99 S28-S31.
Glatt C E, Snowman A M, Sibley D R, et al, 1995, Clozapine selective labeling of sites resembling 5HT6 serotonin receptors may reflect psychoactive profile. Mol. Medicine 1: 398-406.
Goldberg T E, Kelsoe J R, Weinberger D R, Pliskin N H, Kirwin P D, Berman K F, 1988, Performance of schizophrenic patients on putative neuropsychological tests of frontal lobe function. Int J. Neurosci. 42(1-2):51-8.
Goldstein J M, Link B G, 1988, Gender and the expression of schizophrenia. J Psychiatr Res.; 22(2):141-55.
Goldstein J M, Seidman L J, Goodman J M, Koren D, Lee H, Weintraub S, Tsuang M T, 1998, Are there sex differences in neuropsychological functions among patients with schizophrenia? Am J Psychiatry. 155(10):1358-64.
Goldstein J M, Seidman L J, Santangelo S, Knapp P H, Tsuang M T, 1994, Are schizophrenic men at higher risk for developmental deficits than schizophrenic women? Implications for adult neuropsychological functions. J Psychiatr Res. 28(6):483-98.
Green M F, Marshall B D Jr, Wirshing W C, Ames D, Marder S R, McGurk S. Kern R S, Mintz J. 1997, Does risperidone improve verbal working memory in treatment-resistant schizophrenia? Am J Psychiatry. 154(6):799-804.
Hagger C, Buckley P. Kenny J T, Friedman L, Ubogy D, Meltzer H Y, 1993, Improvement in cognitive functions and psychiatric symptoms in treatment-refractory schizophrenic patients receiving clozapine. Biol Psychiatry. 34(10):702-12.
Hoyer D, Engel G and HO Kalkman, 1985, Molecular pharmacology of 5-HT1 and 5-HT2 recognition sites in rat and pig brain membranes: radioligand binding studies with [3H]5-HT, [3H]8-OH-DPAT, (−)[125I]iodocyanopindolol, [3H]mesulergine and [3H]ketanserin. Eur J. Pharmacol. 118(1-2):13-23.
Hoyer D, Pazos A, Probst A and J M Palacios, 1986, Serotonin receptors in the human brain. I. Characterization and autoradiographic localization of 5-HT1A recognition sites. Apparent absence of 5-HT1B recognition sites. Brain Res. 376(1):85-96.
Hoyer D, Pazos A, Probst A and J M Palacios, 1986, Serotonin receptors in the human brain. II. Characterization and autoradiographic localization of 5-HT1C and 5-HT2 recognition sites. Brain Res. 376(1):97-107.
Hoyer D, Srivatsa S, Pazos A, Engel G and J M Palacios, 1986, [125I]LSD labels 5-HT1C recognition sites in pig choroid plexus membranes. Comparison with [3H]mesulergine and [3H]5-HT binding. Neurosci Lett. 69(3):269-74.
Javitt D C, 2001, Management of negative symptoms of schizophrenia. Curr Psychiatry Rep. 3(5):413-7.
Kanba S, Suzuki E, Nomura S, et al, 1994, Affinity of neuroleptics for D₁ receptor of human brain striatum. J. Psych. Neurosci., 19: 265-269.
Kane J M, Woerner M G, Pollack S, et al, 1993, Does clozapine cause tardive dyskinesia? J. Clin. Psychiatry 54: 327-330.
Kay S R, Opler L A, Spitzer R L, Williams J B, Fiszbein A, Gorelick A, 1991, SCID-PANSS: two-tier diagnostic system for psychotic disorders. Compr Psychiatry. 32(4):355-61.
Kongsamut S, Roehr J E, Cai J, et al, 1996, Iloperidone binding to human and rat dopamine and 5-HT receptors. Eur. J. Pharmacol. 317:417-423.
Kurachi M, Matsui M, Kiba K, Suzuki M, Tsunoda M, Yamaguchi N, 1994, Limited visual search on the WAIS Picture Completion test in patients with schizophrenia. Schizophr Res. 12(1):75-80.
Leucht S, Pitschel-Walz G, Abraham D, Kissling W, 1999, Efficacy and extrapyramidal side-effects of the new antipsychotics olanzapine, quetiapine, risperidone, and sertindole compared to conventional antipsychotics and placebo. A meta-analysis of randomised controlled trials. Schizophr Res. 35(1):51-68.
Lewis D A, Lieberman J A, 2000, Catching up on schizophrenia: natural history and neurobiology. Neuron 28(2):325-34.
Lewis D A, Pierri J N, Volk D W, Melchitzky D S, Woo T U, 1999, Altered GABA neurotransmission and prefrontal cortical dysfunction in schizophrenia. Biol Psychiatry. 46(5):616-26.
Lewis R, Bagnall A, Leitner M, 2000, Sertindole for schizophrenia. Cochrane Database Syst Rev. 2:CD001715.
Leysen J, 2000, Novel Atypical Antipsychotics, Atypical Antipsychotics, Eds. B A Ellenbroek and A R Cools, Series Eds. M J Parnham and J Bruinvels, Birkhauser Verlag, Basel-Boston-Berlin.
Leysen J E, Gommeren W, Eens A, de Chaffoy de Courcelles D, Stoof J C, Janssen P A, 1988, Biochemical profile of risperidone, a new antipsychotic. J Pharmacol Exp Ther. 247(2):661-70.
Leysen J E, Gommeren W, Mertens J, et al, 1993, Comparison of in vitro binding properties of a series of dopamine antagonists and agonists for cloned human dopamine D2S and D2L receptors and for D₂ receptors in rat striatal and mesolimbic tissues, using [125I] 2′-iodospiperone. Psychopharmacol. 110: 27-36.
Lieberman J A, 1993, Understanding the mechanism of action of atypical antipsychotic drugs. a review of compounds in use and development. Brit. J. Pharmacol. 163: 7-18.
Mahurin R K, Velligan D I, Miller A L, 1998, Executive-frontal lobe cognitive dysfunction in schizophrenia: a symptom subtype analysis. Psychiatry Res. 79(2):139-49.
Malmberg A, Jackson D M, Eriksson A M, et al, 1993, Unique Binding Characteristics of Antipsychotic Agents Interacting with Human Dopamine D_{2A}, D_{2B}, and D₃ Receptors. Mol. Pharmacol. 43: 749-754.
Matz R, Rick W, Oh D et al, 1974, Clozapine-A potential antipsychotic agent without extrapyramidal manifestations. Curr. Ther. Research 16: 687-695.
Meltzer H Y, 1994, An overview of the mechanism of action of clozapine. J Clin Psychiatry. 55 Suppl B:47-52.
Meltzer H Y, 1996, Pre-clinical pharmacology of atypical antipsychotic drugs: a selective review. Brit. J. Psychiatry 168: 23-31.
Meltzer H Y, 1998, Suicide in schizophrenia: risk factors and clozapine treatment. J Clin Psychiatry 59 Suppl 3:15-20.
Meltzer H Y, 1999, The role of serotonin in antipsychotic drug action. Neuropsychopharmacology. 21(2 Suppl):106S-115S.
Meltzer H Y, Bastani B, Young K K, et al, 1989a, A prospective study of clozapine in treatment-resistant schizophrenic patients. Psychopharmacol. 238: 332-339.
Meltzer H Y, Matsubara S and Lee J-C, 1989b, Classification of typical and atypical antipsychotic drugs on the basis of dopamine D-1, D-2 and serotonin pKi values. J. Pharmacol. Exp. Ther. 251: 238-246.
Meltzer H Y, Matsubara S, Lee J C, 1989, The ratios of serotonin2 and dopamine2 affinities differentiate atypical and typical antipsychotic drugs. Psychopharmacol Bull. 25(3):390-2.
Meltzer H Y, McGurk S R, 1999, The effects of clozapine, risperidone, and olanzapine on cognitive function in schizophrenia. Schizophr Bull. 25(2):233-55.
Meltzer H Y, Thompson P A, Lee M A, Ranjan R, 1996, Neuropsychologic deficits in schizophrenia: relation to social function and effect of antipsychotic drug treatment. Neuropsychopharmacology. 14(3 Suppl):27S-33S.
Meltzer, H Y, Atypical Antipsychotic Drugs, 2000, Psychopharmacology—The Fourth Generation of Progress, The American College of Neuropsychopharmacology website: http://www.acnp.org/g4/4thgen.php
Niswender C M, Herrick-Davis K, Dilley G E, Meltzer H Y, Overholser J C, Stockmeier C A, Emeson R B, Sanders-Bush E, 2001, RNA editing of the human serotonin 5-HT2C receptor. alterations in suicide and implications for serotonergic pharmacotherapy. Neuropsychopharmacology. 24(5):478-91.
O'Donovan M C, Owen M J, 1999, Candidate-gene association studies of schizophrenia. Am J Hum Genet. 65(3):587-92.
Olney J W, Newcomer J W, Farber N B, 1999, NMDA receptor hypofunction model of schizophrenia. J Psychiatr Res. 33(6):523-33.
Pandey G N, 1997, Altered serotonin function in suicide. Evidence from platelet and neuroendocrine studies. Ann N Y Acad. Sci. 836:182-200.
Pazos A and J M Palacios, 1985, Quantitative autoradiographic mapping of serotonin receptors in the rat brain. I. Serotonin-1 receptors. Brain Res. 346(2):205-30.
Pazos A, Hoyer D and J M Palacios, 1985, The binding of serotonergic ligands to the porcine choroids plexus: characterization of a new type of serotonin recognition site. Eur J. Pharmacol. 106:539-546.
Pazos A, Probst A, and J M Palacios, 1987, Serotonin receptors in the human brain—III. Autoradiographic mapping of serotonin-1 receptors. Neurosci. 21(1):97-122.
Pezawas L, Quiner S, Moertl D, Tauscher J, Barnas C, Kufferle B, Wolf R, Kasper S, 2000, Efficacy, cardiac safety and tolerability of sertindole: a drug surveillance. Int Clin Psychopharmacol. 15(4):207-14.
Pickar D, Rubinow K, 2001, Pharmacogenomics of psychiatric disorders. Trends Pharmacol Sci. 22(2):75-83.
Pilowski L S, Costa D C, Ell P J, et al, 1992, Clozapine, single photon emission tomography, and the D2 dopamine receptor blockade hypothesis of schizophrenia. Lancet 340: 199-202.
Portnoff L A, Yesavage J A, Acker M B, 1981, Visual search performance by paranoid and chronic undifferentiated schizophrenics. Percept Mot Skills 53(2):411-8.
Purdon S E, Jones B D, Stip E, Labelle A, Addington D, David S R, Breier A, Tollefson G D, 2000, Neuropsychological change in early phase schizophrenia during 12 months of treatment with olanzapine, risperidone, or haloperidol. The Canadian Collaborative Group for research in schizophrenia. Arch Gen Psychiatry. 57(3):249-58.
Purdon S E, Malla A, Labelle A, Lit W, 2001, Neuropsychological change in patients with schizophrenia after treatment with quetiapine or haloperidol. J Psychiatry Neurosci. 26(2):137-49.
Reyntjens A, Gelders Y G, Hoppenbrouwers-M-LJA, Vanden-Bussche G, 1986, Thymosthenic effects of ritanserin (R 55667), a centrally acting serotonin-S-2 receptor blocker. Drug Dev. Research 8(1-4):205-211.
Richelson E and Nelson A, 1984, Antagonism by neuroleptics of neurotransmitter receptors of normal human brain in vitro. Eur. J. Pharmacol. 103: 197-204.
Richelson E, 1996, Preclinical pharmacology of neuroleptics: focus on new generation compounds. J. Clin. Psychiatry 57: 4-11.
Roth B L, Ciaranello R D and Meltzer H Y, 1992, Binding of typical and atypical antipsychotic agents to transient expressed 5-HT_{1C} receptors. J. Pharmacol. Exp. Ther. 260, 1361-1365.
Roth B L, Craigo S C, Choudhary M S, et al, 1994, Binding of typical and atypical antipsychotic agents to 5-hydroxy-tryptamine-6 and 5-hydroxytryptamine-7 receptors. J. Pharmacol. Exp. Ther. 268(3):1403-1408.
Roth B L, Tandra S, Burgess L H, et al, 1995, D₄ Dopamine receptor binding affinity does not distinguish between typical and atypical antipsychotic drugs. Psychopharmacol. 120: 365-368.
Sauriol L, Laporta M, Edwardes M D, Deslandes M, Ricard N, Suissa S, 2001, Meta-analysis comparing newer antipsychotic drugs for the treatment of schizophrenia: evaluating the indirect approach. Clin Ther. 23(6):942-56.
Schotte A, Janssen P F M, Gommeren W, et al, 1996, Risperidone compared with new and reference antipsychotic drugs: in vitro and in vivo receptor binding. Psychopharmacol. 124: 57-73.
Seeger T F, Seymour P A, Schmidt A W, et al, 1995, Ziprasidone (CP-88, 059): a new antipsychotic with combined dopamine and serotonin receptor antagonist activity. J. Pharmacol. Exp. Ther. 275: 101-113.
Seeman P and Lee T, 1975, Antipsychotic drugs: direct correlation between clinical potency and presynaptic action on dopamine neurons. Science 188: 1217-1219.
Siris S G, 2001, Suicide and schizophrenia. J Psychopharmacol. 15(2):127-35.
Szewczak M R, Corbett R, Rush D K, et al, 1995, The pharmacological profile of iloperidone, a novel atypical anti-psychotic agent. J. Pharmacol. Exp. Ther. 274: 1404-1413.
Taylor D M, McAskill R, 2000, Atypical antipsychotics and weight gain—a systematic review. Acta Psychiatr Scand. 101(6):416-32.
Tran P V, Hamilton S H, Kuntz A J, Potvin J H, Andersen S W, Beasley C Jr, Tollefson G D, 1997, Double-blind comparison of olanzapine versus risperidone in the treatment of schizophrenia and other psychotic disorders. J Clin Psychopharmacol. 17 (5):407-18.
Tsuang M T, Stone W S, Faraone S V, 2001, Genes, environment and schizophrenia. Br J Psychiatry Suppl. 40:S18-24.
van Tol HHM, Bunzow J R, Guan H-C, et al, 1991, Cloning of the gene for a human dopamine D4 receptor with high affinity for the antipsychotic clozapine. Nature 350: 610-615.
Waddington J L and J F Quinn, 2000, From first to second generation antipsychotics. Atypical Antipsychotics, Eds. BA Ellenbroek and AR Cools, Series Eds. MJ Parnham and J Bruinvels, Birkhauser Verlag, Basel-Boston-Berlin, pp: 19-33
Velligan D I, Newcomer J, Pultz J, Csernansky J, Hoff A L, Mahurin R, Miller A L, 2002, Does cognitive function improve with quetiapine in comparison to haloperidol? Schizophr Res. 53(3):239-48.
Wander T J, Nelson A, Okazaki H, et al, 1987, Antagonism by neuroleptics of serotonin 5-HT1A and 5-HT₂ receptors of normal human brain in vitro. Eur J. Pharmacol. 143: 279-282.
Weinberger D R, 2000, Neurodevelopmental Perspectives on Schizophrenia, Psychopharmacology—The Fourth Generation of Progress, The American College of Neuropsychopharmacology website: http://www.acnp.org/g4/4thgen.php
Wetterling T, 2001, Bodyweight gain with atypical antipsychotics. A comparative review. Drug Saf. 24(1):59-73.
Wirshing D A, Wirshing W C, Kysar L, Berisford M A, Goldstein D, Pashdag J, Mintz J, Marder S R, 1999, Novel antipsychotics: comparison of weight gain liabilities. J Clin Psychiatry 60(6):358-63.
Yagaloff K A, Lozano G, Van Dyke T, Levine A and P R Hartig, 1986, Serotonin 5-HT1C receptors are expressed at high density on choroid plexus tumors from transgenic mice. Brain Res. 385(2):389-94.

1-19. (canceled)

20. A method for determining the suitability of a candidate compound for use in the treatment of negative symptoms of and/or cognitive dysfunction in schizophrenia, refractory schizophrenia, suicidality or mild cognitive impairment which comprises:
a) assessing the affinity of the compound at the 5-HT2C receptor;
b) assessing the affinity of the compound at at least two other major sites of said compound interaction;
c) applying the assessed affinities to the following formula:
$$\frac{X}{A} + \frac{X}{B} = Y$$
wherein: X is the affinity of a compound for interaction at the 5-HT2C receptor and A and B are the average affinity values of a compound for interaction at two major sites other than the 5-HT2C receptor;
and selecting compounds in which Y≧1.80 as suitable compounds for the treatment of cognitive dysfunction in and/or negative symptoms of schizophrenia, refractory schizophrenia, suicidality or mild cognitive impairment, provided that:
(a) for the treatment of cognitive dysfunction in and/or negative symptoms of schizophrenia or refractory schizophrenia, the compound selected is other than ritanserin, clozapine, fluperlapine, loxapine, ORG-5222, pipamperone, sertindole, olanzapine, zotepine or ziprasidone;
(b) for the indications cognitive dysfunction in schizophrenia or mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and
(c) for the treatment of schizophrenic suicidality, the compound selected is other than clozapine.

21. The method of claim 20 in which A and B are different and are independently selected from the group consisting of the 5-HT_{1A}, 5-HT_{2A}, 5-HT₃, 5-HT₆, 5-HT₇, D₁, D₂-S, D₂-L, D₃, D₄, D₅ M₁, M₂, M₃, M₄, M₅, mACh, α₁, α₂, H₁ or sigma receptors.

22. The method of claim 21 in which A is the value for affinity at the 5-HT2A receptor.

23. The method of claim 21 in which B is the value for affinity at the D2 receptor.

24. The method of claim 20 in which the compound selected has Y≧2.00.

25. A therapeutic product comprising a 5-HT2C receptor antagonist and a typical anti-psychotic, said product being a combined preparation for the treatment of schizophrenia, suicidality or mild cognitive impairment, wherein said antagonist and said anti-psychotic are administered simultaneously, separately or sequentially.

26. A therapeutic product in which the 5-HT2C receptor antagonist is identifiable by the method of claim 20.

27. A method for the treatment of a patient suffering from symptoms associated with a condition selected from the group consisting of negative symptoms of schizophrenia, cognitive dysfunction in schizophrenia, refractory schizophrenia, suicidality and mild cognitive impairment with a pharmaceutically effective amount of a compound having a relative 5-HT2C affinity of ≧1.80, wherein the relative 5HT2C affinity is determined according to the method of claim 20 with the proviso that:
(a) when the condition is selected from the group consisting of negative symptoms of schizophrenia, cognitive dysfunction in schizophrenia and refractory schizophrenia, the compound is other than ritanserin, clozapine, fluperlapine, loxapine, ORG-5222, pipamperone, sertindole, olanzapine, zotepine or ziprasidone;
(b) when the condition is selected from the group consisting of cognitive dysfunction in schizophrenia and mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and
(c) when the condition is schizophrenic suicidality, the compound is other than clozapine.

28. A method according to claim 27 wherein the condition is refractory schizophrenia, with the proviso that the compound is other than ritanserin, clozapine, fluperlapine, loxapine, ORG-5222, pipamperone, sertindole, olanzapine, zotepine or ziprasidone.

29. A method according to claim 27 wherein the condition is suicidality, with the proviso that, when the suicidality is in a schizophrenic patient, the compound is other than clozapine.

30. A method according to claim 29, wherein the suicidality is in a schizophrenic patient.

31. A method according to claim 27 wherein the condition is mild cognitive impairment with the proviso that the compound is other than deramciclane or N-desmethylderamciclane.

32. A method according to claim 27 wherein the compound is as described in a publication selected from the group consisting of WO 97/16429, WO 97/44334, U.S. Pat. No. 5,010,078, EP 161,218, EP401,707, EP526,434, DE02834114, EP 210,893, U.S. Pat. No. 3,580,916, U.S. Pat. No. 5,043,341, EP 620,222, EP 208,235, EP 437,790, DE 02614406, U.S. Pat. No. 4,338,317, EP 271,013, EP 110,435, EP 398,326, WO 92/05170, WO 95/01976, WO 96/23783, WO 98/04289, WO 97/48700, WO 00/48602, WO 00/26186, WO 99/58490, WO 99/52517, WO 99/51237, WO 99/46245, WO 99/43319, WO 99/33841, WO 99/33840, WO 99/25356, WO 99/09017, WO 99/03833, WO 99/00119, WO 98/56367, WO 98/52943, WO 98/50358, WO 98/50346, WO 98/50343, WO 98/41527, WO 98/38165, WO 98/30561, WO 98/30546, WO 98/24785, WO 98/21958, WO 98/04261, WO 97/48699, WO 97/41858, WO 97/39001, WO 97/37989, WO 97/20845, WO 97/12880, WO 97/08167, WO 97/06155, WO 97/00872, WO 96/39382, WO 96/30366, WO 96/24351, WO 96/23769, WO 96/18629, WO 96/14320, WO 96/11930, WO 96/11929, WO 96/02537, WO 95/29177, WO 95/25731, WO 95/24194, WO 95/21844, WO 95/18117, WO 95/12591, WO9 94/22871, WO 94/18958, WO 94/18182, WO 94/18170, WO 94/14801, WO 94/04533, WO 94/02462, WO 93/18028, WO 93/18026, WO 93/16081, WO 93/16051, WO 93/14758, WO 93/12790, WO 92/15302, WO 92/10192, WO 91/18602, WO 01/68585, WO 01/68067, WO 01/52855, WO 01/38329, WO 01/26621, WO 01/25229, WO 01/19371, WO 00/76984, WO 00/68181, WO 00/63185, WO 00/62782, WO 00/61129, WO 00/61128, WO 00/37068, WO 00/06165, U.S. Pat. No. 6,143,325, U.S. Pat. No. 5,854,248, U.S. Pat. No. 5,739,336, U.S. Pat. No. 5,693,645, U.S. Pat. No. 5,674,875, U.S. Pat. No. 5,498,618, U.S. Pat. No. 5,371,093, U.S. Pat. No. 5,266,571, U.S. Pat. No. 5,116,852, U.S. Pat. No. 5,106,855, U.S. Pat. No. 5,030,656, U.S. Pat. No. 5,013,735, U.S. Pat. No. 4,985,352, U.S. Pat. No. 4,914,107, U.S. Pat. No. 4,914,100, U.S. Pat. No. 4,906,639, U.S. Pat. No. 4,902,691, U.S. Pat. No. 4,891,376, U.S. Pat. No. 4,847,261, JP 13220375, JP 12204040, JP 11171865, JP 11080155, JP 10316634, JP 10077271, JP 09040646, JP 08053416, JP 08040999, JP 07228573, JP 07179337, JO 00158067, GB 02303303, GB 02301774, EP 01118610, EP 1070716, EP 01052245, EP 01000944, EP 00905136, EP 00797995, EP 00797994, EP 00769297, EP 00749971, EP 00749967, EP 00718299, EP 00700905, EP 00686393, EP 00682015, EP 0661266, EP 00657426, EP 006554440, EP 00613898, EP 00596449, EP 00559569, EP 00545120, EP 00522226, EP 00511074, EP 00511073, EP 00493687, EP 00484988, EP 00465398, EP 00452074, EP 00389352, EP 00388081, EP 00384228, EP 00379308, EP 00378468, EP 00375297, EP 00374042, EP 00373998, EP 00363963, EP 00354030, EP 00337136, EP 00332528, EP 00320983, EP 00218433 and EP 00145494.

33. A method according to claim 27 in which the compound is selected from the group consisting of AHR-16303B (AH Robins Co. Inc), AP-792 and AT-1015 (Ajinomoto Co. Inc.), BMS-181102 (Bristol Myers Squibb), CV-5197 (Takeda Chemical Industries Ltd), dotarizine (Ferrer Internacional SA), E-2101 (Eisai Co Ltd), eltoprazine (Solvay SA), emopamil (Knoll AG), HT-90B (Chugai Pharmaceutical Co Ltd), ICI-169369 and ICI-170809 (Zeneca Group plc), LU-26042 and LU-29066 (H Lundbeck A/S), NPC-18166 (Scios Inc), Org-38457 (NV Organon), pelanserin (Cinvestav), perbufylline (Siegfried Group), SB-206553 and SB-242084 (SmithKline Beecham), SR-46615A (Sanofi Recherche SA), SUN-9221 (Suntory Ltd) tropoxin (Russian Academy Medical Science) and YM-992 (Yamanouchi Pharmaceutical Co Ltd).

34. A method according to claim 27 in which the compound is selected from the group consisting of Ro-60-0759, RS-102221, SDZ-SER-082, ICI-169369, deramciclane, N-desmethyl-deramciclane, amesergide, sergolexole, CGS-18102A and LU-26042.

35. A method according to claim 34 in which the compound is selected from the group consisting of deramciclane, N-desmethyl-deramciclane, amesergide, sergolexole, CGS-18102A and LU-26042.

36. A method according to claim 27 wherein the condition is suicidality or mild cognitive impairment and wherein the compound is selected from the group consisting of ritanserin, clozapine, fluperlapine, loxapine, ORG-5222, pipamperone, sertindole, olanzapine, zotepine and ziprasidone, with the proviso that when the suicidality is in a schizophrenic patient, the compound is other than clozapine.

37. A method for the treatment of a patient suffering from symptoms associated with a condition selected from the group consisting of refractory schizophrenia, suicidality and mild cognitive impairment with a pharmaceutically effective amount of a 5-HT2C receptor antagonist with the proviso that:
(a) when the condition is refractory schizophrenia, the 5-HT2C receptor antagonist is other than ritanserin, clozapine, fluperlapine, loxapine, ORG-5222, pipamperone, sertindole, olanzapine, zotepine or ziprasidone;
(b) when the condition is mild cognitive impairment, the 5-HT2C receptor antagonist is other than (1R,2S,4R)-(−)-2-phenyl-2-(dimethylaminoethoxy)-1,7,7-trimethyl-bicyclo[2.2.1]heptane, (1R,2S,4R)-(−)-2-phenyl-2-(methylaminoethoxy)-1,7,7-trimethyl-bicyclo[2.2.1]heptane and pharmaceutically acceptable acid addition salts thereof; and
(c) when the condition is schizophrenic suicidality, the 5-HT2C receptor antagonist is other than clozapine.

38. A method according to claim 37 wherein the condition is refractory schizophrenia, with the proviso that the antagonist is other than ritanserin, clozapine, fluperlapine, loxapine, ORG-5222, pipamperone, sertindole, olanzapine, zotepine or ziprasidone.

39. A method according to claim 37 wherein the condition is suicidality, with the proviso that, when the suicidality is in a schizophrenic patient, the 5-HT2C receptor antagonist is other than clozapine.

40. A method according to claim 39, wherein the suicidality is in a schizophrenic patient.

41. A method according to claim 37 wherein the condition is mild cognitive impairment with the proviso that the antagonist is other than deramciclane or N-desmethylderamciclane.

42. A method according to claim 37 wherein the 5-HT2C receptor antagonist is as described in a publication selected from the group consisting of WO 97/16429, WO 97/44334, U.S. Pat. No. 5,010,078, EP 161,218, EP 401,707, EP 526,434, DE 02834114, EP210,893, U.S. Pat. No. 3,580,916, U.S. Pat. No. 5,043,341, EP620,222, EP208,235, EP 437,790, DE 02614406, U.S. Pat. No. 4,338,317, EP 271,013, EP 110,435, EP 398,326, WO 92/05170, WO 95/01976, WO 96/23783, WO 98/04289, WO 97/48700, WO 00/48602, WO 00/26186, WO 99/58490, WO 99/52517, WO 99/51237, WO 99/46245, WO 99/43319, WO 99/33841, WO 99/33840, WO 99/25356, WO 99/09017, WO 99/03833, WO 99/00119, WO 98/56367, WO 98/52943, WO 98/50358, WO 98/50346, WO 98/50343, WO 98/41527, WO 98/38165, WO 98/30561, WO 98/30546, WO 98/24785, WO 98/21958, WO 98/04261, WO 97/48699, WO 97/41858, WO 97/39001, WO 97/37989, WO 97/20845, WO 97/12880, WO 97/08167, WO 97/06155, WO 97/00872, WO 96/39382, WO 96/30366, WO 96/24351, WO 96/23769, WO 96/18629, WO 96/14320, WO 96/11930, WO 96/11929, WO 96/02537, WO 95/29177, WO 95/25731, WO 95/24194, WO 95/21844, WO 95/18117, WO 95/12591, WO9 94/22871, WO 94/18958, WO 94/18182, WO 94/18170, WO 94/14801, WO 94/04533, WO 94/02462, WO 93/18028, WO 93/18026, WO 93/16081, WO 93/16051, WO 93/14758, WO 93/12790, WO 92/15302, WO 92/10192, WO 91/18602, WO 01/68585, WO 01/68067, WO 01/52855, WO 01/38329, WO 01/26621, WO 01/25229, WO 01/19371, WO 00/76984, WO 00/68181, WO 00/63185, WO 00/62782, WO 00/61129, WO 00/61128, WO 00/37068, WO 00/06165, U.S. Pat. No. 6,143,325, U.S. Pat. No. 5,854,248, U.S. Pat. No. 5,739,336, U.S. Pat. No. 5,693,645, U.S. Pat. No. 5,674,875, U.S. Pat. No. 5,498,618, U.S. Pat. No. 5,371,093, U.S. Pat. No. 5,266,571, U.S. Pat. No. 5,116,852, U.S. Pat. No. 5,106,855, U.S. Pat. No. 5,030,656, U.S. Pat. No. 5,013,735, U.S. Pat. No. 4,985,352, U.S. Pat. No. 4,914,107, U.S. Pat. No. 4,914,100, U.S. Pat. No. 4,906,639, U.S. Pat. No. 4,902,691, U.S. Pat. No. 4,891,376, U.S. Pat. No. 4,847,261, JP 13220375, JP 12204040, JP 11171865, JP 11080155, JP 10316634, JP 10077271, JP 09040646, JP 08053416, JP 08040999, JP 07228573, JP 07179337, JO 00158067, GB 02303303, GB 02301774, EP 01118610, EP 1070716, EP 01052245, EP 01000944, EP 00905136, EP 00797995, EP 00797994, EP 00769297, EP 00749971, EP 00749967, EP 00718299, EP 00700905, EP 00686393, EP 00682015, EP 0661266, EP 00657426, EP 006554440, EP 00613898, EP 00596449, EP 00559569, EP 00545120, EP 00522226, EP 00511074, EP 00511073, EP 00493687, EP 00484988, EP 00465398, EP 00452074, EP 00389352, EP 00388081, EP 00384228, EP 00379308, EP 00378468, EP 00375297, EP 00374042, EP 00373998, EP 00363963, EP 00354030, EP 00337136, EP 00332528, EP 00320983, EP 00218433 and EP 00145494.

43. A method according to claim 37 in which the 5-HT2C receptor antagonist is selected from the group consisting of AHR-16303B (AH Robins Co. Inc), AP-792 and AT-1015 (Ajinomoto Co. Inc.), BMS-181102 (Bristol Myers Squibb), CV-5197 (Takeda Chemical Industries Ltd), dotarizine (Ferrer Internacional SA), E-2101 (Eisai Co Ltd), eltoprazine (Solvay SA), emopamil (Knoll AG), HT-90B (Chugai Pharmaceutical Co Ltd), ICI-169369 and ICI-170809 (Zeneca Group plc), LU-26042 and LU-29066 (H Lundbeck A/S), NPC-18166 (Scios Inc), Org-38457 (NV Organon), pelanserin (Cinvestav), perbufylline (Siegfried Group), SB-206553 and SB-242084 (SmithKline Beecham), SR-46615A (Sanofi Recherche SA), SUN-9221 (Suntory Ltd) tropoxin (Russian Academy Medical Science) and YM-992 (Yamanouchi Pharmaceutical Co Ltd).

44. A method according to claim 37 in which the 5-HT2C receptor antagonist is selected from the group consisting of Ro-60-0759, RS-102221, SDZ-SER-082, ICI-169369, deramciclane, N-desmethyl-deramciclane, amesergide, sergolexole, CGS-18102A and LU-26042.

45. A method according to claim 44 in which the 5-HT2C receptor antagonist is selected from the group consisting of deramciclane, N-desmethyl-deramciclane, amesergide, sergolexole, CGS-18102A and LU-26042.

46. A method according to claim 37 wherein the condition is suicidality or mild cognitive impairment and wherein the 5-HT2C receptor antagonist is selected from the group consisting of ritanserin, clozapine, fluperlapine, loxapine, ORG-5222, pipamperone, sertindole, olanzapine, zotepine and ziprasidone, with the proviso that when the suicidality is in a schizophrenic patient, the 5-HT2C receptor antagonist is other than clozapine.

47. A product according to claim 25 in which the 5-HT2C receptor antagonist is as described in a publication selected from the group consisting of WO 97/16429, WO 97/44334, U.S. Pat. No. 5,010,078, EP 161,218, EP 401,707, EP 526,434, DE 02834114, EP210,893, U.S. Pat. No. 3,580,916, U.S. Pat. No. 5,043,341, EP620,222, EP208,235, EP 437,790, DE 02614406, U.S. Pat. No. 4,338,317, EP 271,013, EP 110,435, EP 398,326, WO 92/05170, WO 95/01976, WO 96/23783, WO 98/04289, WO 97/48700, WO 00/48602, WO 00/26186, WO 99/58490, WO 99/52517, WO 99/51237, WO 99/46245, WO 99/43319, WO 99/33841, WO 99/33840, WO 99/25356, WO 99/09017, WO 99/03833, WO 99/00119, WO 98/56367, WO 98/52943, WO 98/50358, WO 98/50346, WO 98/50343, WO 98/41527, WO 98/38165, WO 98/30561, WO 98/30546, WO 98/24785, WO 98/21958, WO 98/04261, WO 97/48699, WO 97/41858, WO 97/39001, WO 97/37989, WO 97/20845, WO 97/12880, WO 97/08167, WO 97/06155, WO 97/00872, WO 96/39382, WO 96/30366, WO 96/24351, WO 96/23769, WO 96/18629, WO 96/14320, WO 96/11930, WO 96/11929, WO 96/02537, WO 95/29177, WO 95/25731, WO 95/24194, WO 95/21844, WO 95/18117, WO 95/12591, WO9 94/22871, WO 94/18958, WO 94/18182, WO 94/18170, WO 94/14801, WO 94/04533, WO 94/02462, WO 93/18028, WO 93/18026, WO 93/16081, WO 93/16051, WO 93/14758, WO 93/12790, WO 92/15302, WO 92/10192, WO 91/18602, WO 01/68585, WO 01/68067, WO 01/52855, WO 01/38329, WO 01/26621, WO 01/25229, WO 01/19371, WO 00/76984, WO 00/68181, WO 00/63185, WO 00/62782, WO 00/61129, WO 00/61128, WO 00/37068, WO 00/06165, U.S. Pat. No. 6,143,325, U.S. Pat. No. 5,854,248, U.S. Pat. No. 5,739,336, U.S. Pat. No. 5,693,645, U.S. Pat. No. 5,674,875, U.S. Pat. No. 5,498,618, U.S. Pat. No. 5,371,093, U.S. Pat. No. 5,266,571, U.S. Pat. No. 5,116,852, U.S. Pat. No. 5,106,855, U.S. Pat. No. 5,030,656, U.S. Pat. No. 5,013,735, U.S. Pat. No. 4,985,352, U.S. Pat. No. 4,914,107, U.S. Pat. No. 4,914,100, U.S. Pat. No. 4,906,639, U.S. Pat. No. 4,902,691, U.S. Pat. No. 4,891,376, U.S. Pat. No. 4,847,261, JP 13220375, JP 12204040, JP 11171865, JP 11080155, JP 10316634, JP 10077271, JP 09040646, JP 08053416, JP 08040999, JP 07228573, JP 07179337, JO 00158067, GB 02303303, GB 02301774, EP 01118610, EP 1070716, EP 01052245, EP 01000944, EP 00905136, EP 00797995, EP 00797994, EP 00769297, EP 00749971, EP 00749967, EP 00718299, EP 00700905, EP 00686393, EP 00682015, EP 0661266, EP 00657426, EP 006554440, EP 00613898, EP 00596449, EP 00559569, EP 00545120, EP 00522226, EP 00511074, EP 00511073, EP 00493687, EP 00484988, EP 00465398, EP 00452074, EP 00389352, EP 00388081, EP 00384228, EP 00379308, EP 00378468, EP 00375297, EP 00374042, EP 00373998, EP 00363963, EP 00354030, EP 00337136, EP 00332528, EP 00320983, EP 00218433 and EP 00145494.

48. A product according to claim 25 in which the 5-HT2C receptor antagonist is selected from the group consisting of AHR-16303B (AH Robins Co. Inc), AP-792 and AT-1015 (Ajinomoto Co. Inc.), BMS-181102 (Bristol Myers Squibb), CV-5197 (Takeda Chemical Industries Ltd), dotarizine (Ferrer Internacional SA), E-2101 (Eisai Co Ltd), eltoprazine (Solvay SA), emopamil (Knoll AG), HT-90B (Chugai Pharmaceutical Co Ltd), ICI-169369 and ICI-170809 (Zeneca Group plc), LU-26042 and LU-29066 (H Lundbeck A/S), NPC-18166 (Scios Inc), Org-38457 (NV Organon), pelanserin (Cinvestav), perbufylline (Siegfried Group), SB-206553 and SB-242084 (SmithKline Beecham), SR-46615A (Sanofi Recherche SA), SUN-9221 (Suntory Ltd) tropoxin (Russian Academy Medical Science) and YM-992 (Yamanouchi Pharmaceutical Co Ltd).

49. A product according to claim 25 in which the 5-HT2C receptor antagonist is selected from the group consisting of Ro-60-0759, RS-102221, SDZ-SER-082, ICI-169369, deramciclane, N-desmethyl-deramciclane, amesergide, sergolexole, CGS-18102A and LU-26042.

50. A product according to claim 25 in which the 5-HT2C receptor antagonist is selected from the group consisting of deramciclane, N-desmethyl-deramciclane, amesergide, sergolexole, CGS-18102A and LU-26042.

51. A product according to claim 25 in which the 5-HT2C receptor antagonist is selected from the group consisting of ritanserin, clozapine, fluperlapine, loxapine, ORG-5222, pipamperone, sertindole, olanzapine, zotepine and ziprasidone, with the proviso that when the suicidality is in a schizophrenic patient, the 5-HT2C receptor antagonist is other than clozapine.
